(19) United States
(12) Patent Application Publication
(10) Pub. No.: US 2015/0367004 A1
(43) Pub. Date: Dec. 24, 2015

(54) COMPOSITIONS AND METHODS OF DIAGNOSING OCULAR DISEASES

(71) Applicant: MOLECULAR TARGETING TECHNOLOGIES, INC., West Chester, PA (US)

(72) Inventors: Y. Koon Pak, Malvern, PA (US); Man Kwong Kwong, Los Angeles, CA (US); Richard W. Yee, Houston, TX (US); Kai Chen, Grabiel, CA (US); Brian D. Gray, Exton, PA (US)

(21) Appl. No.: 14/650,995

(22) PCT Filed: Nov. 21, 2013
(86) PCT No.: PCT/US2013/071136

§ 371 Date: Jun. 10, 2015

Related U.S. Application Data
(60) Provisional application No. 61/735,223, filed on Dec. 10, 2012.

(51) Int. Cl. A61K 51/04; A61K 49/00

(52) U.S. Cl. CPC A61K 51/0478; A61K 51/0497; A61K 49/0052

(57) ABSTRACT

The invention provides novel multi-modality probes for pathologic cell tracking which allow labeling of dying cells with new probes and tracking them via non-invasive imaging techniques to diagnose ocular diseases, determine disease progression and evaluate effectiveness of treatment. The molecular probes of the invention can be topically, locally, or systemically administered for diagnosing and monitoring improvement or progression of any ocular diseases.

### PRIORITY CLAIMS AND RELATED PATENT APPLICATIONS

This application claims the benefit of priority from U.S. Provisional Application Ser. No. 61/735,223, filed on Dec. 10, 2012, the entire content of which is incorporated herein by reference in its entirety.

### TECHNICAL FIELDS OF THE INVENTION

The invention generally relates to compositions and methods for diagnosing and/or monitoring ocular disorders. More particularly, the invention relates to compositions and methods for diagnosing and/or monitoring ocular disorders associated with cell death using Bis(zinc (II)-dipicolylamine) (Zn-DPA) and derivatives and analogs thereof.

### BACKGROUND OF THE INVENTION

Many diseases of the eye cause pathological cellular changes in the eye associated adnexa, i.e., eyelids and meibomian glands and ocular innervation. Glaucoma, acute ischemic optic neuropathy, macular degeneration, retinal dystrophies (e.g., retinitis pigmentosa), retinal detachment, retinal tears or holes, ischemic retinopathies, optic neuropathies, diabetic retinopathy, retinal dystrophies, dry eye disease, dysfunctional tear disease, ocular surface disease, lid margin disease (e.g., anterior and posterior blepharits), graft versus host disease, corneal dystrophy (e.g., Fuchs dystrophy), ocular infection, ocular inflammation, ocular transplant, ocular surgeries and complications cause injury or death of retinal neurons, vascular cells, and ocular cells, optic nerve cells, which can lead to deterioration and loss of vision and/or ocular damage and/or ocular discomfort. For example, primary open-angle glaucoma (POAG) is a progressive disease leading to optic nerve damage and ultimately blindness. The cause of POAG has been the subject of extensive studies for many years, but is still not fully understood. Glaucoma results in the neuronal degeneration of the retina and optic nerve. Even under optimal medical care and surgical treatment, it is still associated with a gradual loss of retinal ganglion cells (RGCs), which causes a decline of visual function. (Van Buskirk, et al. 1993 Am J Ophthalmol. 116:636-40; Schumer, et al. 1994 Arch Ophthalmol. 112:37-44.)

Cell death is an important biological process that plays a critical physiological and pathological role within an organism. Programmed cell death, a highly regulated process involving various initiators, effectors and executors, plays important roles not only during development but also under pathological conditions. Disruption of homeostasis, either by excessive or deficient cell death, is a hallmark of many pathological conditions including cancer, neurodegenerative disorders, cardiovascular diseases, autoimmune diseases, infectious and metabolic diseases and ocular disorders. (Thompson 1995 Science 267:1456-62; van Heerde, et al. 2000 Cardiovasc Res. 45:549-59.)

Cell death is typically divided into two distinctive processes, apoptosis and necrosis; however, there is increasing evidence for additional pathways such as autophagy, mitotic catastrophe and senescence. (Okada, et al. 2004 Nat Rev Cancer 4:592-603; Marx 2006 Science. 312:1160-1; Blum, et al. 2012 Science 335:970-3; Dixon, et al. 2012 Cell 149:1060-72.)

Necrotic cells exhibit increased cytoplasmic vacuolation, organelle degeneration, condensation of chromatin into irregular patches, and an increase in cell volume that results in irreversible rupture of the plasma membrane.

Apoptotic cells are characteristically scattered throughout the tissue and show condensation of chromatin underlying the nuclear membrane and reduction of nuclear size and cell volume. Biochemically, apoptotic cell death includes activation of caspases, mitochondrial outer membrane permeabilization, DNA fragmentation, generation of reactive oxygen species (ROS), lysosomal membrane permeabilization (LMP), and exposure of molecular biomarkers such as phosphatidylserine (PS) on the outer leaflet of the plasma membrane. Unambiguous assignment of a cell death process is a challenging task, since different death process can induce the same change in one biomarker. For example, PS exposure, ROS generation, and LMP are biomarkers associated with both apoptosis and necrosis. (Krysko, et al. 2004 Apoptosis 9:495-500; Festjens, et al. 2006 Biochim Biophys Acta 1757:1371-87; Galluzzi, et al. 2007 Cell Death Differ. 14:1237-43.) There is also evidence for crosstalk between multiple death processes that are occurring simultaneously. (Maiuri, et al. 2007 Nat Rev Mol Cell Biol. 8:741-52.) There may be a continuum between necrosis and apoptosis and which cell death process the cell decides to undergo may depend on the disease nature and intensity. (Portera-Cailliau, et al. 1997 J Comp Neurol. 378:70-87.)

Cell death has been implicated in major ocular diseases that lead to blindness including ARMD, glaucoma, cataract, diabetic retinopathy, and retinal detachment, and additionally, ocular disorders that lead to visual impairment and discomfort including dry eye disease, evaporative dry eye, lid margin disease, blepharitis, ocular graft versus host disease, corneal dystrophy, ocular infection, corneal transplant, cornea rejection, ocular surgeries and etc. (Nickel's, et al. 1996 Ophthalmic Genet. 17:145-65; Carella 2003 Eur J Ophthalmol. 13:S5-10.) Once cell death occurs, the cells disappear and are lost. The tissue loses the visual function and sometimes some ocular cells cannot be replaced such as neurons and corneal endothelia cells. For example, the loss of retinal ganglion cells in glaucoma patients, the loss of photoreceptors in ARMD cause visual field defects and ultimately blindness and the loss of corneal endothelial cells in Fuchs dystrophy cause corneal edema and loss of corneal transparency.

Diagnosis and monitoring of ocular diseases is briefly divided into subjective and objective examinations. The findings of subjective examination are very variable and less consistent because it heavily relies on the response from the patients. Objective examination may not detect the early stage of an ocular disease and is difficult to monitor the rate of progress. For example, optic nerve head cupping can be visualized by ophthalmoscopy or measured by technologies using Optical Coherence Tomography (OCT) in a glaucoma patient after a group of axons are lost in a region. Statistical visual field abnormalities could be only detected by automated perimetry (one of the standard visual function testes to diagnose glaucoma) after more than 20%-35% retinal ganglion cells are already lost in patients, and a change of 5 dB visual threshold could be measured after 25% of retinal ganglion cells are lost. (Kerrigan-Baumrind, et al. 2000 Invest Ophthalmol Vis Sci. 41:741-8.) Currently available examinations have limitations in diagnosing and monitoring ocular diseases. Another ocular disease that is inadequately diagnosed and sequentially difficult to monitor and has poor sign and symptom correlation is dry eye or ocular surface disease.

The ability to identify markers that allow for objectively tracking of pathogenic cells and disease as well as age-related degenerative progression will allow the potential to objectively correlate more efficiently better correlation of sign and symptoms and the early diagnosis of diseases that otherwise would not be diagnosed during the early phases of the onset of the pathogenic process.

Labeling and tracking of dying cells have the potential to dramatically change the diagnosis of ocular diseases and/or provide more information to confirm the diagnosis of an ocular disease. The current diagnostic tests cannot detect early change in ocular disease and so that are not sensitive to monitor the rate of progression and change after therapy. Critical insights can be achieved by observing the dying cells in vivo overtime using noninvasive imaging techniques. Currently, no diagnostic tool has been available to diagnose ocular disorders by the actual tracking of affected and dying ocular cells in human.

To date, multiple modalities, including optical imaging, ultrasound, nuclear imaging, computed tomography and various MRI techniques are increasingly being used to investigate the new insightful markers applications at the cellular and molecular level, both in the basic science and clinical science research investigations respectively. The eye is particularly suitable for direct molecular imaging due to its uniquely easy optical accessibility.

An emerging technology that allows for visualization of interactions between molecular probes and biological targets is molecular imaging, which can be divided into two general categories: (1) the direct labeling method, and (2) the reporter gene approach. The former involves using an image-detectable probe that can be loaded into cells either intra or extracellularly during tracking. This method does not involve extensive cell manipulation and therefore is preferred for clinical application. Limitations include marker dilution upon cell division, making the cells eventually invisible; and marker labels effluxing from cells or degrading over time.

The direct labeling methods include antibody labeling, protein expression (labeling of proteins, peptides, oligonucleotides), measuring enzyme activity, nanoparticle approaches and markers for labeling apoptosis and apoptotic events.

Current apoptotic imaging has evolved from annexin V staining in vitro, which demonstrates apoptotic cells through binding to phosphatidylserines. The molecules are normally found only on the inner surface of plasma membranes and are therefore not accessible to annexin V. During apoptosis, these phosphatidylserines molecules are transiently exposed to the extracellular space, allowing binding of annexin. Whereas in other medical fields applications of annexin-based apoptosis imaging have been developed for SPECT (^{99m}Tc-annexin), PET, MRI and ultrasound, optical imaging has been used to detect dying cells in vivo. (Dumont, et al. 2001 Nat Med. 7:1352-5; Boersma, et al. 2005 J Nucl Med. 46:2035-50.)

Annexin V as a potential candidate as a biomarker is limited. Lederle et al. documented that higher apoptosis rate was detected in treated tumors by TUNEL staining. (Lederle, et al. 2011 EJNMMI Res. 1:26.) However, both imaging using Annexin V and gamma counting using ^{99m}Tc-HYNIC-Annexin V failed in showing higher accumulation in treated tumors. Optical tomography even indicated higher probe accumulation in controls. Vascularization was strongly reduced after antiangiogenic therapy such as Avastin, demonstrated by contrast-enhanced ultrasound, optical imaging, and immunohistology. This group addressed that the failure of annexin-based apoptosis assessment in vivo can be explained by the breakdown of the vasculature after therapy, resulting in reduced probe/tracer delivery. In addition, the disadvantages of Annexin V limit its application on humans and living subjects such as: (i) poor penetration due to large molecule; (ii) poor penetration through the blood brain barrier; (iii) poor elimination and degradation; and (iv) binding is Ca²⁺ dependent.

Thus, there remains an unmet need for novel probes for pathologic cell tracking in the eye. In particular, methods for labeling dying cells with probes and tracking them via non-invasive imaging techniques are strongly desired.

### SUMMARY OF THE INVENTION

This invention relates to multimodality probes for imaging, tracking and analyzing dying cells, related biological samples, and methods of use. The invention provides the use of novel probes for pathologic cell tracking which allow labeling of dying cells with new probes and tracking them via non-invasive imaging techniques to diagnose ocular diseases, determine disease progression and evaluate effectiveness of treatment. The molecular probes of the invention can be topically, locally, or systemically administered for diagnosing disease and improvement or progression of any ocular diseases. For example, in vivo and ex vivo data of the optical probes demonstrated their utility for detecting the presence of dying cells which result from diseases leading to the loss of vision, discomfort and other signs and symptoms of ocular diseases.

In one aspect, the invention generally relates to a method for diagnosing an ocular disease characterized by an increase in the extracellular level of phosphatidylserine in a human or living subject. The method includes: (a) administering an effective amount of a single or dual-modality compound comprising a fluorescence moiety with or without a radioactive moiety, a linker, and an anionic surface-targeting moiety; and (b) measuring the fluorescent emission of the fluorescent compound to obtain an image, thereby determining the site of an ocular disease,

(c) measuring the radioactive emission of the radioactive moiety if present to obtain an image, thereby determining the site of an ocular disease, and (d) measuring both the fluorescent emission of the fluorescent compound and radioactive emission of the radioactive moiety if present to obtain an image, thereby determining the site of an ocular disease. The anion surface-targeting moiety comprises an organometallic complex.

The method may further include: repeating step (b) at selected intervals wherein the repeating is effective to track changes in the intensity of fluorescent/radioactive emission in the subject over time to detect changes either in location or in number of cells that undergo cell death.

In another aspect, the invention generally relates to a kit for diagnosing an ocular disease characterized by an increase in the extracellular level of phosphatidylserine in a human or living subject, comprising an effective amount of a compound comprising a fluorescence moiety, a linker, and an anionic surface-targeting moiety comprising an organometallic complex.

In another aspect, the invention generally relates to new composition of matter involving multimodality probes.

In yet another aspect, the invention generally relates to a pharmaceutical composition comprising a compound having the structure:

(F)-------(R)--------(Y)-------Z

wherein (F) is a fluorescent moiety, (R) is a radioactive moiety; (Y) is a linking moiety, (Z) is an anionic surface-targeting entity, and wherein the anion surface-targeting moiety (Z) comprises an organometallic complex.

### BRIEF DESCRIPTION OF THE DRAWINGS

The following drawings form part of the present specification and are included to further demonstrate certain in vivo aspects of the present invention. The invention may be better understood by reference to these micrographs in combination with the detailed description of specific embodiments presented herein.

FIG. 1. Labeling of Zn-DPA 480 on corneal cells at 1 day after benzalkonium chloride (BAC)-induced corneal toxicity in the rats. Zn-DPA 480 was topically administered on the corneal surface.

FIG. 2. Labeling of Zn-DPA 480 on apoptotic cells after BAC-induced corneal toxicity in the rats. Zn-DPA 480 was topically administered on the corneal surface. Apoptotic cells were labeled by in situ TdT-mediated biotin-dUTP nick end labeling (TUNEL).

FIG. 3. In vivo imaging of Zn-DPA 480 after BAC-induced corneal toxicity in the rats. A gradient of corneal damages was induced by topical administration of 0.2, 0.1 and 0.02% BAC for 7 days. Zn-DPA 480 was topically administered on the corneal surface. External biomicroscopic examination and lissamine green staining were compared.

FIG. 4. Ex vivo analysis of Zn-DPA 480 and apoptosis on cornea after BAC-induced toxicity in the rats. The ocular tissues obtained from FIG. 3 were examined by histopathological studies and the co-localization of Zn-DPA 480 and TUNEL for apoptosis was evaluated.

FIG. 5. Labeling of Zn-DPA 480 and Zn-DPA rhodamine on retinal ganglion cells 24 hours after N-methyl-D-aspartate (NMDA)-induced apoptosis. Retrograde labeling dye, fluoro-gold, was used to specifically label retinal ganglion cells. Zn-DPA 480 and Zn-DPA rhodamine was intravenously (systemically) administered into the rats.

FIG. 6. Labeling of Zn-DPA 480 and Zn-DPA rhodamine in the whole mount retinas after NMDA-induced apoptosis in the rats. Zn-DPA 480 and Zn-DPA rhodamine was intravenously administered into the rats. Animals were euthanized 2 and 6 hours after NMDA injection.

FIG. 7. Labeling of Zn-DPA 480 in the whole mount retina before and after NMDA-induced apoptosis. Solution of Zn-DPA 480 was injected intravitreally (locally) 1 hour prior to euthanasia. Animals were euthanized 4 and 48 hours after NMDA injection.

FIG. 8. Quantitative analysis of Zn-DPA 480 on retinal ganglion cells and glial cells in the retinas after NMDA-induced apoptosis. Animals were euthanized at 24 hours (Peak of apoptosis) after NMDA injection. Zn-DPA 480 was injected intravitreally 1 hour prior to euthanasia. Immunohistochemistry using antibodies against Rbpms (retinal ganglion cell marker), vimentin (glial cell marker) and beta-tubulin (retinal ganglion cell marker) were performed. The percentage of double labeling of Zn-DPA 480 with vimentin and beta-tubulin was determined.

FIG. 9. Labeling of Zn-DPA 480 on retinal ganglion cells after optic nerve axotomy in the rats. Retrograde labeling dye, DTMR, was implanted to pre-label retinal ganglion cells. Solution of Zn-DPA 480 was injected intravenously 2 hours prior to euthanasia.

FIG. 10. Cell uptake assay: time-dependent uptake of 8b (dual functionality; red solid line) and 17b (negative control; blue dotted line) in U87MG cells treated with 50 nM of Paclitaxel for 16 h (n=3, mean±SD).

FIG. 11. Decay-corrected whole-body coronal microPET images of nude mice bearing U87MG tumor (n=5/group) at 1, 2, and 4 hours post-injection of 8b (7.4 MBq), with tumors indicated by arrows (FIG. 11A), and ex vivo PET imaging of tumor and normal tissues of 8b after euthanizing the mice at 4 hours post-injection (FIG. 11B).

### DETAILED DESCRIPTION OF THE INVENTION

The invention provides novel probes for pathologic cell tracking which allow labeling of dying cells with new probes and tracking them via non-invasive imaging techniques to diagnose ocular diseases, determine disease progression and evaluate effectiveness of treatment. The molecular probes of the invention can be topically, locally, or systemically administered for diagnosing and monitoring improvement or progression of any ocular disease. Labeling dying cells with new probes and tracking them via non-invasive imaging techniques may hold the key to diagnosing an ocular disease, determining the rate of disease progress and evaluating the effectiveness of treatment.

Programmed cell death, a highly regulated process involving various initiators, effectors and executors, plays important roles not only during development but also under pathological conditions. Many pathological changes in the eye, such as glaucoma, acute ischemic optic neuropathy, macular degeneration, retinitis pigmentosa, retinal detachment, retinal tears or holes, ischemic retinopathies, optic neuropathies, diabetic retinopathy, retinal dystrophies, dry eye, corneal dystrophy, ocular infection, ocular transplant, ocular surgeries and complications cause injury or death of retinal neurons, vascular cells, and ocular cells, which can lead to deterioration and loss of vision and/or ocular discomfort.

Labeling and tracking of dying cells have the potential to dramatically change the diagnosis of ocular diseases and provide more information to confirm the diagnosis of an ocular disease. Critical insights can be achieved by observing the dying cells in vivo overtime using noninvasive imaging techniques. At present, no diagnostic tool is available to diagnose ocular disorders by tracking dying ocular cells in human and living subjects.

The eye is particularly suitable for molecular imaging due to its unique optical access. Optical molecular imaging technologies use light emitted through fluorescence and the target reporter molecule can be excited by particular wavelength resulting in the emission of light that can be visualized and recorded by camera such as charge-coupled device camera. This technique can be incorporated into available diagnostic instruments including the biomicroscope (slit lamp), optical coherence tomography, ocular coherence microscopy, confocal laser scanning ophthalmoscope, adaptive optics scanning laser ophthalmoscope, ophthalmoscope and fundus camera.

The phospholipid bilayer surrounding animal cells is a dynamic environment made up of four principle phospholipid components, phosphatidylcholine (PC), phosphatidylethanolamine (PE), phosphatidylserine (PS), and sphingomyolin (SM). These four phospholipids are distributed between the two monolayers of the membrane in an asymmetrical fashion, with the choline-containing lipids, PC and SM, largely populating the extracellular leaflet, while the aminophospholipids, PE and especially PS, are restricted primarily to the inner membrane leaflet. This membrane asymmetry is maintained by a family of translocase enzymes. Regardless of the initiating stimulus, the loss of the phospholipid asymmetry inherent to healthy animal cell membranes is a hallmark of apoptosis.

During apoptosis, the PS normally found exclusively on the inner membrane monolayer becomes scrambled between the two membrane leaflets. PS is the only anionic phospholipid component of the plasma membrane, that is, present in appreciable levels, and externalization of PS results in a net buildup of anionic charge on the membrane surface.

PS externalization precedes the up-regulation of protease activity in the cytosol and occurs long before membrane permeabilization and DNA fragmentation begins. Those steps are usually considered as irreversible steps and the cells cannot return to normal. Additionally, the detection of PS externalization avoids the complications of other assays that require access to the cytosol.

As disclosed herein, the application of Zn-DPA and derivatives and analogs thereof provides site-specific in vivo imaging of apoptotic tissue and cells that assist the diagnosis of various diseases including ocular diseases and future treatment. For example, Zn-DPA and related compounds that preferentially bind the PS headgroup can detect the externalized PS on the cell surface.

In one aspect, the invention generally relates to a method for diagnosing an ocular disease characterized by an increase in the extracellular level of phosphatidylserine in a human or living subject. The method includes: (a) administering an effective amount of a compound comprising a fluorescence moiety (F), a radioactive moiety (R), a linker (Y), and an anionic surface-targeting moiety (Z) linked to the fluorescent moiety (F) through the linker (Y);

(F)-----(R)-----(Y)-----(Z)

and (b) measuring the fluorescent emission of the compound to obtain an image if no radioactive moiety is present, thereby determining the site of an ocular disease, (c) measuring the radioactive emission of the compound to obtain an image if no fluorescent moiety is present, thereby determining the site of an ocular disease, (d) measuring both the radioactive emission and fluorescent emission of the compound to obtain an image, thereby determining the site of an ocular disease. The anion surface-targeting moiety comprises an organometallic complex.

The method may further include: repeating step (b) at selected intervals wherein the repeating is effective to track changes in the intensity of fluorescent emission in the subject over time to detect changes either in location or in number of cells that undergo cell death.

In certain embodiments, the ocular disease is a front of the eye disease. In certain embodiments, the ocular disease is a back of the eye disease.

In certain embodiments, the organometallic complex comprises a metal cation complexed to one or more aryl ligands. In certain preferred embodiments, the metal cation is a zinc or copper cation.

In certain embodiments, the fluorescent moiety has an emission max range of about 440 nm-about 900 nm (e.g., about 500 nm, 550 nm, 600 nm, 650 nm, 700 nm, 750 nm, 800 nm, 850 nm) and an absorbance max range of about 380 nm-about 880 nm (e.g., about 400 nm, 450 nm, 500 nm, 550 nm, 600 nm, 650 nm, 700 nm, 750 nm, 800 nm, 850 nm).

The fluorescent moiety may be any suitable fluorescent group, for example, a fluorescein, a rhodamine, boron-dipyrromethene (BODIPY), Cy-3, Cy-5, Cy-7, a squaraine rotaxane, an NIR dye, or a derivative thereof.

The radionuclide may be any suitable radionuclide, for example, ¹⁸F, ⁶⁴Cu, ⁶⁸Ga, ^{99m}Tc, ¹¹¹In, ¹²³I, ¹²⁴I, ⁹⁰Y, ¹⁷⁷Lu, ¹¹C, ¹⁴C, ³H, ³²P, ³³P, ¹⁸⁶Re, ¹⁸⁸Re, or ⁸⁶Zr.

The linker may be any suitable linking group, and may be a cyclic or non-cyclic group. In certain embodiments, the linker comprises a heterocyclic moiety. In certain embodiments, the linker comprises a hydrophilic moiety selected from the group consisting of: hydroxyl, carbonyl, sulfonamide, sulfonate, phosphate, aliphatic hydrocarbon, polyethylene glycol moiety, polar amino acid moiety, peptide, sugar mimetic, and sugar moiety.

Exemplary linkers include:

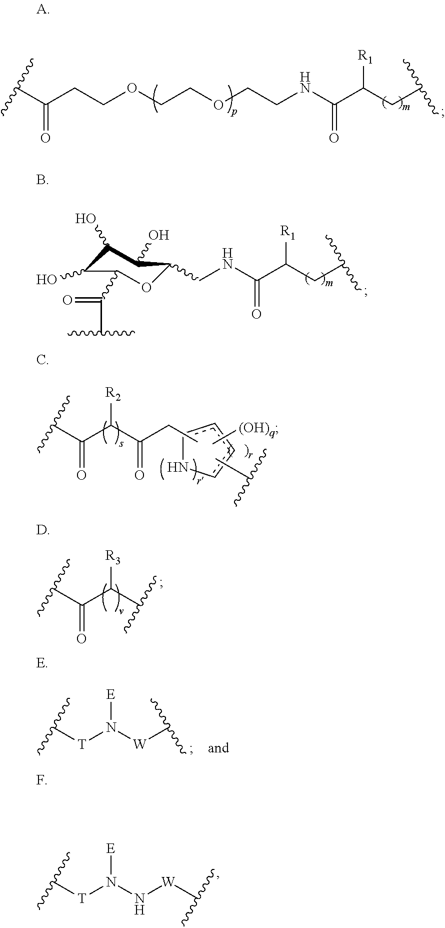

wherein E is:

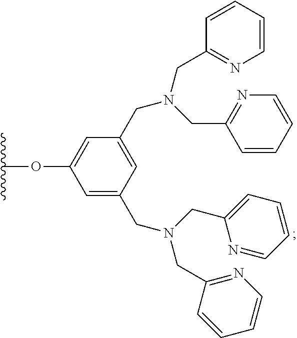

wherein T is selected from the group consisting of:

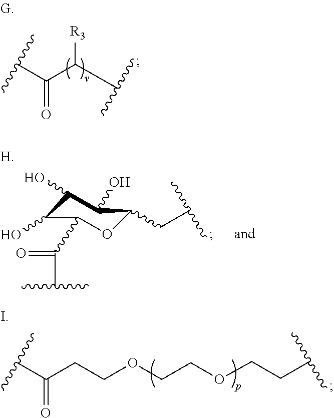

wherein W is selected from the group consisting of:

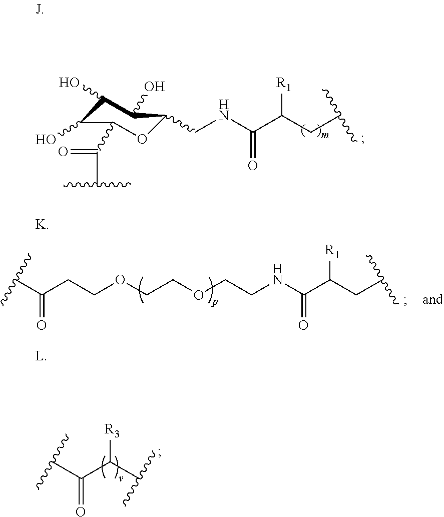

and
wherein
each R₁ is selected from the groups consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, and C₁-C₆-alkoxy-C₁-C₆-alkyl, wherein the alkyl, alkenyl, alkynyl, alkyloxy, aryl, carbocycle, heterocycle, and substitutions thereof;
each R₂ is independently selected from the group consisting of —H, —OH, C₁-C₆ alkyl, C₂-C₆ alkenyl, C₂-C₆ alkynyl, C₁-C₆ alkyloxy, aryl-(C₁-C₆ alkylene)-, hydroxy-C₁-C₆-alkyl, and C₁-C₆-alkoxy-C₁-C₆-alkyl; wherein the alkyl, alkenyl, alkynyl, alkyloxy, and aryl-alkylene groups are each optionally substituted;
each R₃ is independently selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, C₁-C₆-alkoxy-C₁-C₆-alkyl, and a PEG moiety; wherein the alkyl, alkenyl, alkynyl, alkoxy, aryl, carbocycle, and heterocycle groups are each optionally substituted;
   each v is independently selected from 0, 1, 2, 3, and 4;
m is 0, 1, 2, 3 or 4;
p is an integer between 1 and 110;
q is 1, 2, 3 or 4;
r is 1, 2 or 3;
r′ is 0 or 1; and
s is 1, 2, 3 or 4.

In certain preferred embodiments, the anionic surface-targeting moiety binds an anionic phospholipid membrane. In certain preferred embodiments, the anionic surface-targeting moiety binds phosphatidylserine or a structural analog thereof. In certain preferred embodiments, the anionic surface-targeting moiety binds an anionic bacterial surface. The ocular disease involves apoptotic or necrotic cell death.

The compound is administered in any suitable way, including topically, intravenously or systemically.

Exemplary compounds useful in the methods of the invention include:

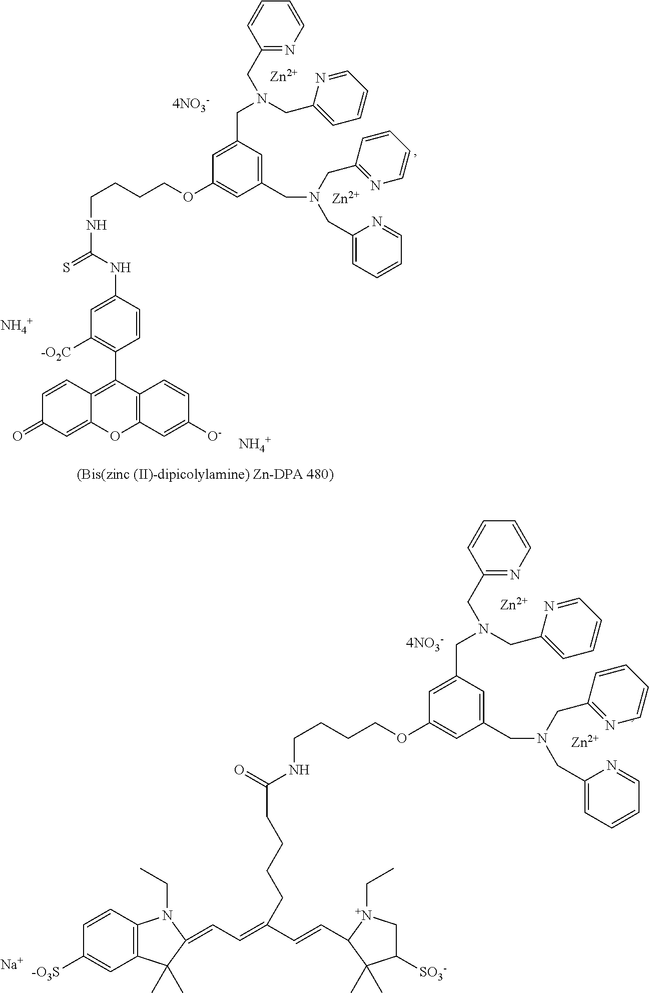

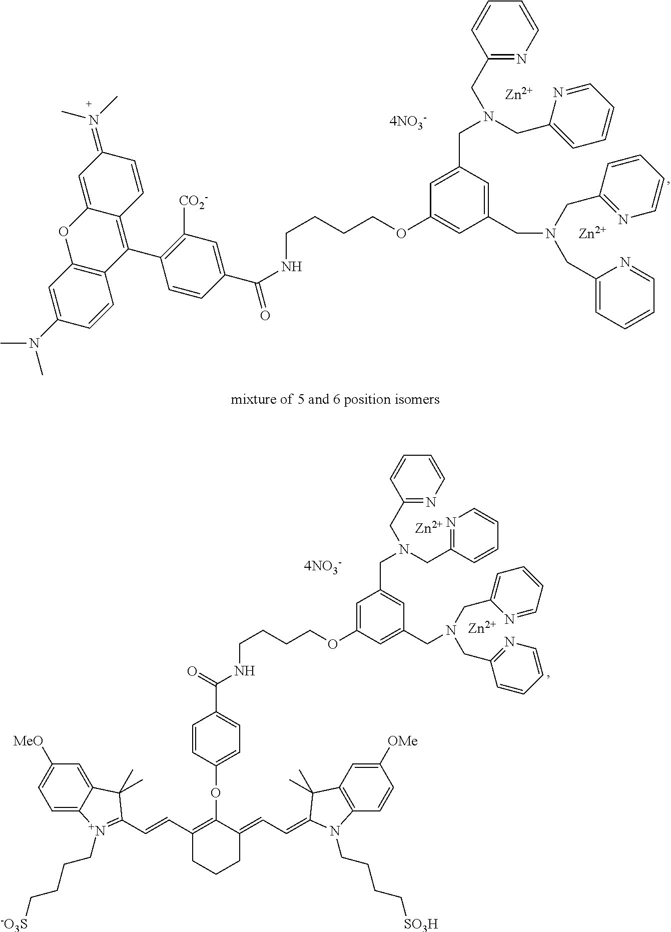

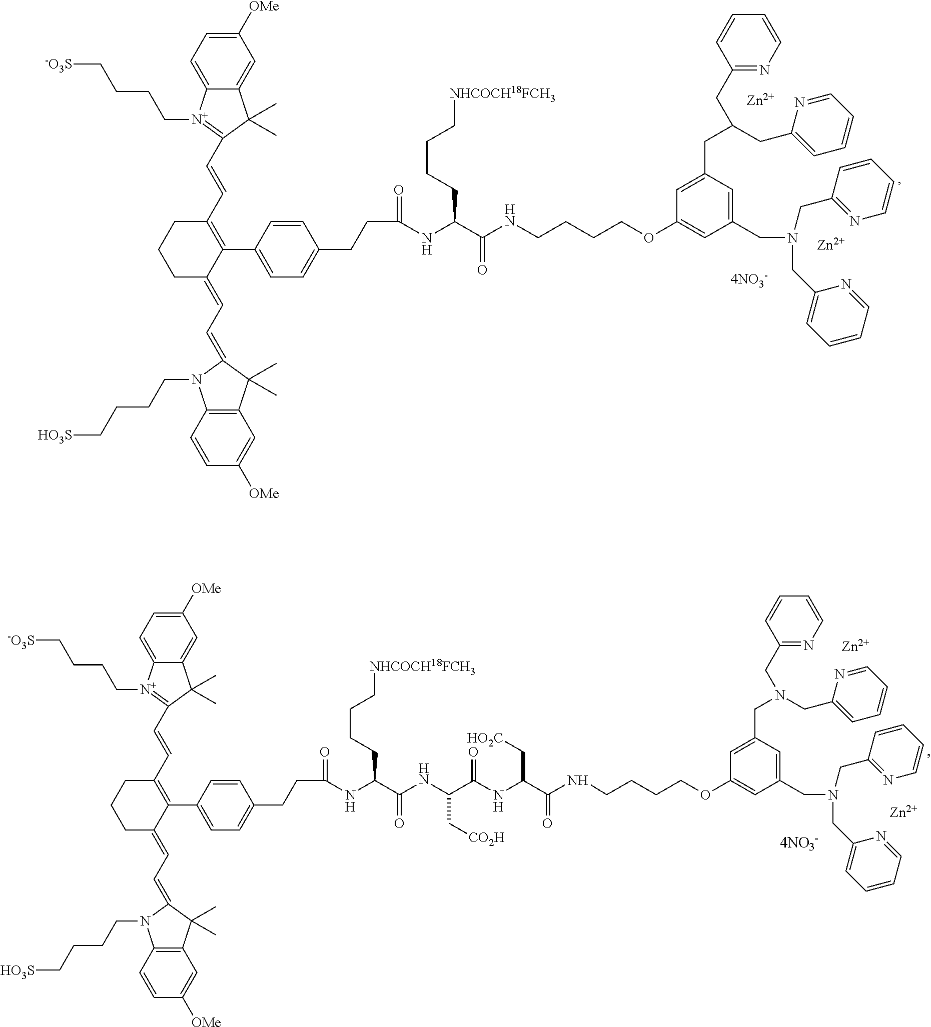

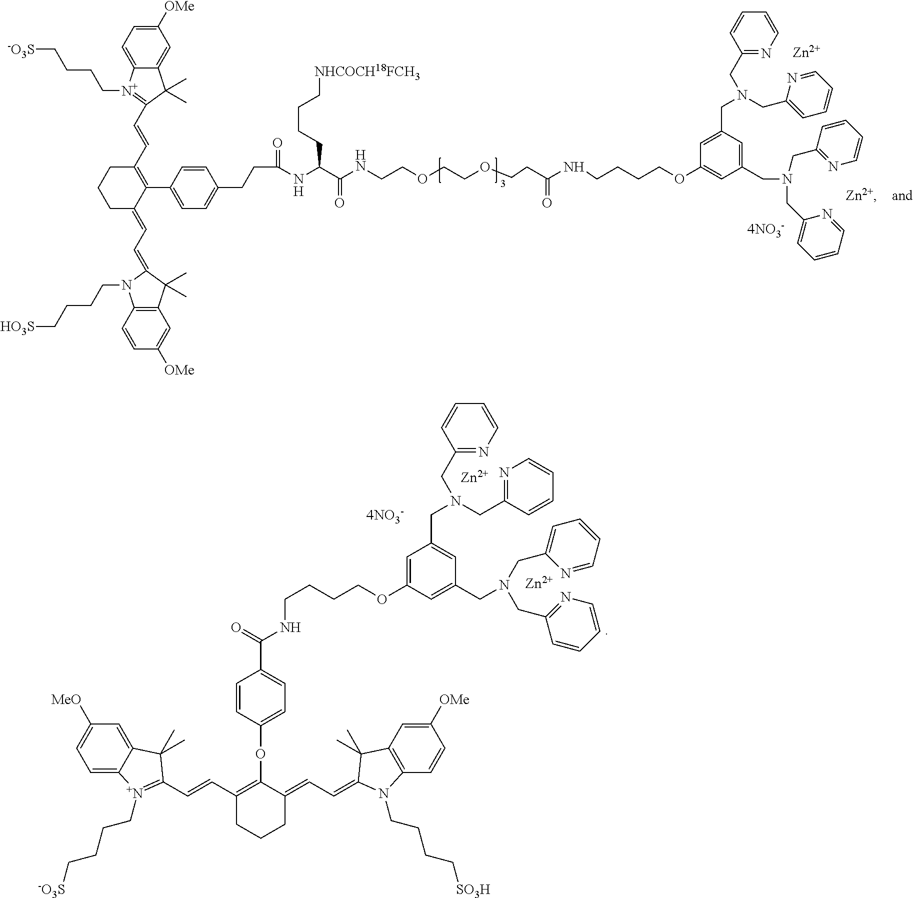

In yet another aspect, the invention generally relates to a kit for diagnosing an ocular disease characterized by an increase in the extracellular level of phosphatidylserine in a human or living subject, comprising an effective amount of a compound comprising a fluorescence moiety (F), a linker (Y), and an anionic surface targeting moiety (Z) comprising an organometallic complex.

In yet another aspect, the invention generally relates to a pharmaceutical composition comprising a compound having the structure:

(F)-------(R)--------(Y)-------Z

wherein (F) is a fluorescent moiety, (R) is a radioactive moiety; (Y) is a linking moiety, (Z) is an anionic surface-targeting entity, and wherein the anion surface-targeting moiety (Z) comprises an organometallic complex.

In certain preferred embodiments, the organometallic complex comprises one or more of zinc or copper cations.

In certain preferred embodiments, the fluorescent moiety (F) has an emission max range of 440 nm-900 nm and an absorbance max range of 380 nm-880 nm.

In certain preferred embodiments, pharmaceutical composition of claim 30, suitable for use in diagnosing an ocular disease characterized by an increase in the extracellular level of phosphatidylserine in a human or living subject.

The application of Zn-DPA provides a number of advantages over fluorescent Annexin V including: (1) fast binding kinetics (DiVittorio, et al. 2006 Org Biomol Chem. 4:1966-76; Koulov, et al. 2003 Cell Death Differ. 10:1357-9.); (2) Ca²⁺ independent binding (Hanshaw et al, 2005); (3) detecting apoptosis in wide variety of conditions (Hanshaw, et al. 2005 Bioorg Med Chem. 13:5035-42); and (4) more intense labeling and smaller molecular size. (Koulov, et al. 2003 Cell Death Differ. 10:1357-9.)

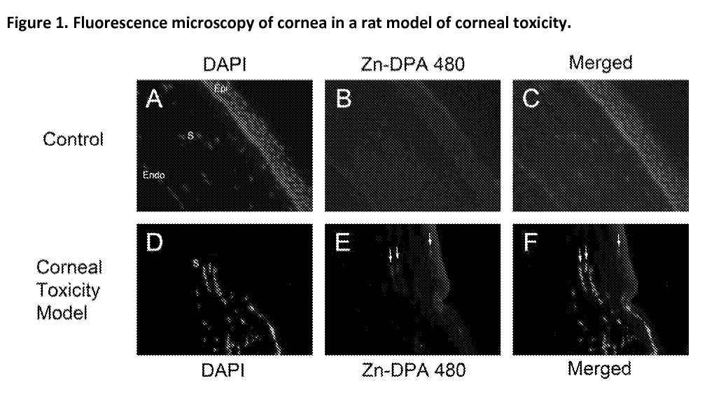

Topical administration of Zn-DPA 480 labeled cells in the cornea as early as 1 day after administration of 2% benzalkonium chloride (BAC), which is known to induce apoptosis in cornea in rodents. The histopathological examination revealed that there was loss of epithelium and stromal cells, and corneal edema. This toxicity effect has been related to dry eye syndrome, preservative-induced ocular discomfort and other ocular surface diseases. The use of Zn-DPA 480 can label not only cells in epithelial layer but also in the stromal layer (mid-cornea) and potentially the endothelial layer when undergoing disease states ie Fuch's corneal endothelial dystrophy. Results are illustrated in FIG. 1.

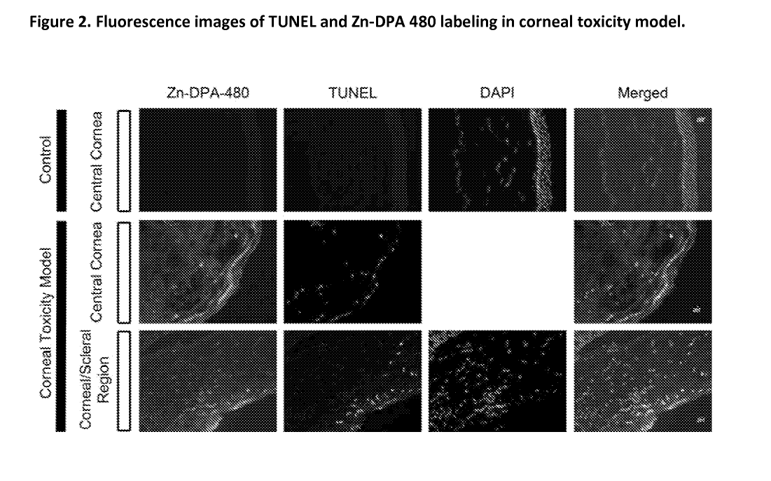

Topical administration of Bis(zinc (II)-dipicolylamine) Zn-DPA 480 labeled apoptotic cells in the cornea 3 days after 0.2% BAC induced toxicity in the rats. The apoptotic cells were labeled by Terminal deoxynucleotidyl transferase dUTP Nick End Labeling (TUNEL) technique, which was used to detect DNA fragmentation, a hallmark of apoptosis. Results are illustrated in FIG. 2.

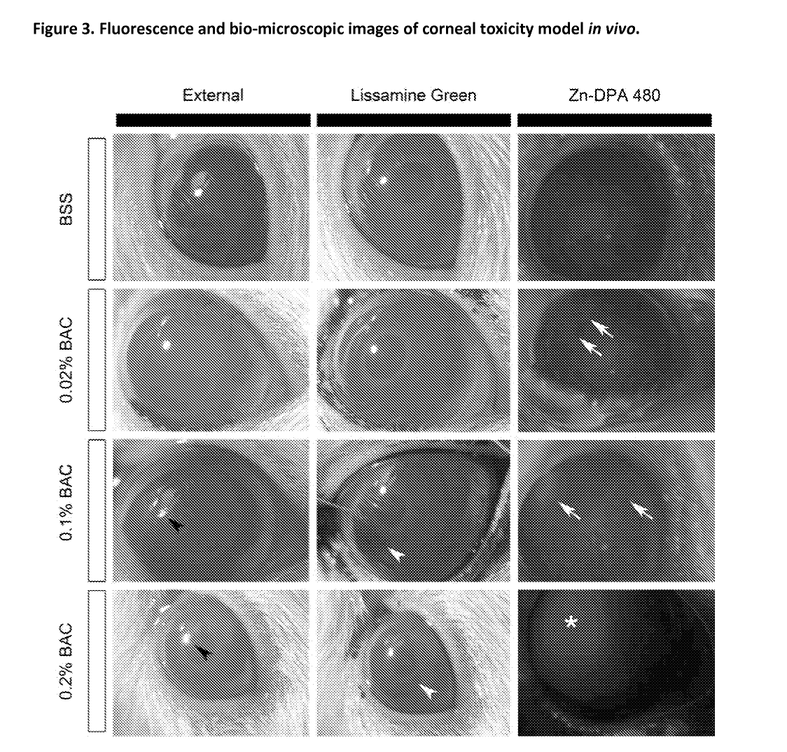

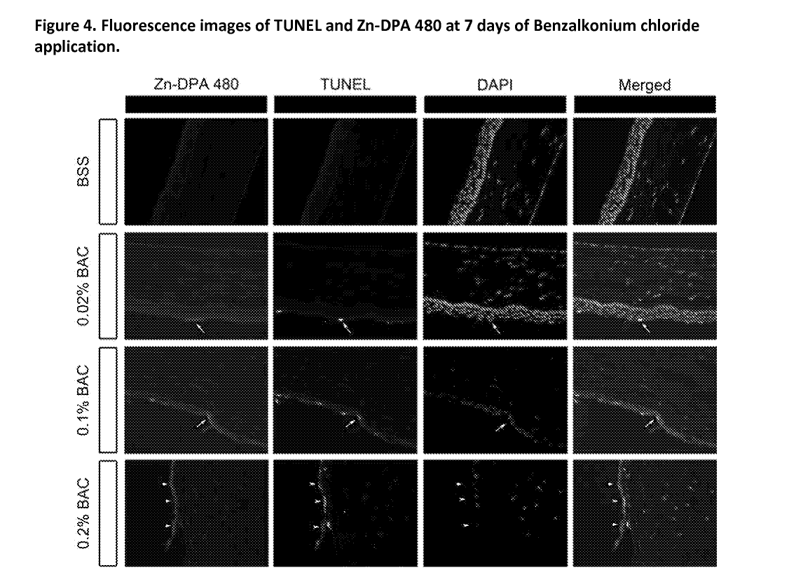

To generate a dose-dependent damage on cornea, solution of 0.2%, 0.1% or 0.02% benzalkonium chloride (BAC) was applied topically on the cornea twice daily for 7 day. Positive punctate staining of Zn-DPA 480 on the corneal surface was noted in cornea after 0.2% BAC-induced toxicity. In vivo imaging of Zn-DPA 480 on cornea after toxicity indicated changes earlier than regular biomicroscopy and lissamine green staining (a standard test for corneal surface diseases). Irregular light reflection and positive lissamine green staining was noted on the cornea after 0.1% and 0.2% BAC administration while in vivo imaging of Zn-DPA 480 showed more intense and extensive labeling in these conditions. Results are illustrated in FIG. 3. The histopathological examination and TUNEL technique showed that Zn-DPA 480 positive cells are TUNEL positive supporting that Zn-DPA 480 labeled cells undergoing apoptosis. Results are illustrated in FIG. 4.

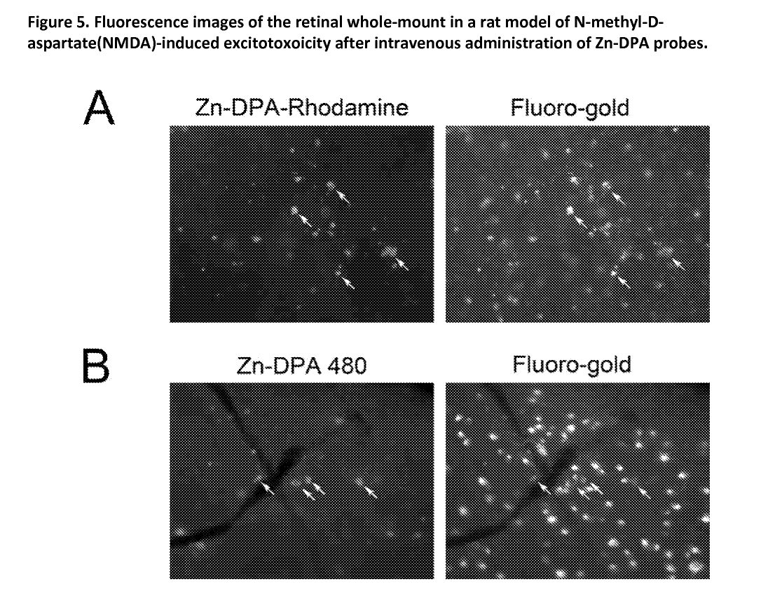

Intravenous administration of Zn-DPA 480 and Zn-DPA rhodamine labeled retinal ganglion cells in the rat retina after NMDA-induced apoptosis, which is related to retinal ganglion cell degeneration in glaucoma. (Lam, et al. 1999 Invest Ophthalmol Vis Sci. 40:2391-2397; Kwong, et al. 2000 Exp Eye Res. 71:437-444; Kwong, et al. 2003 Brain Res. 970(1-2):119-130.) Results are illustrated in FIG. 5.

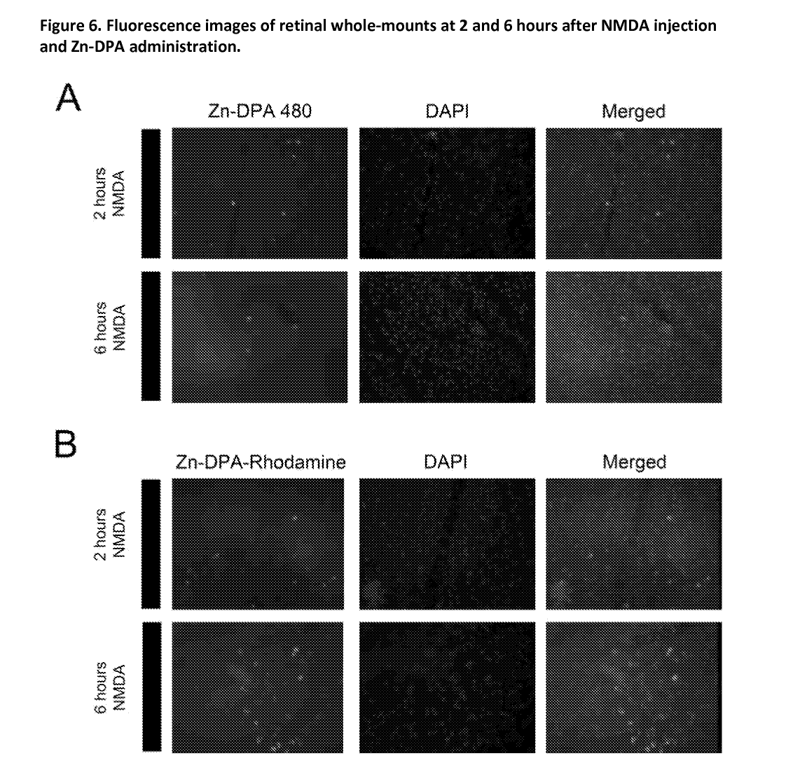

Intravenous administration of Zn-DPA 480 and Zn-DPA rhodamine can label retinal cells as early as 2 hours, which well precedes the peak of DNA fragmentation in this animal model (18-24 hrs after NMDA injection). Results are illustrated in FIG. 6.

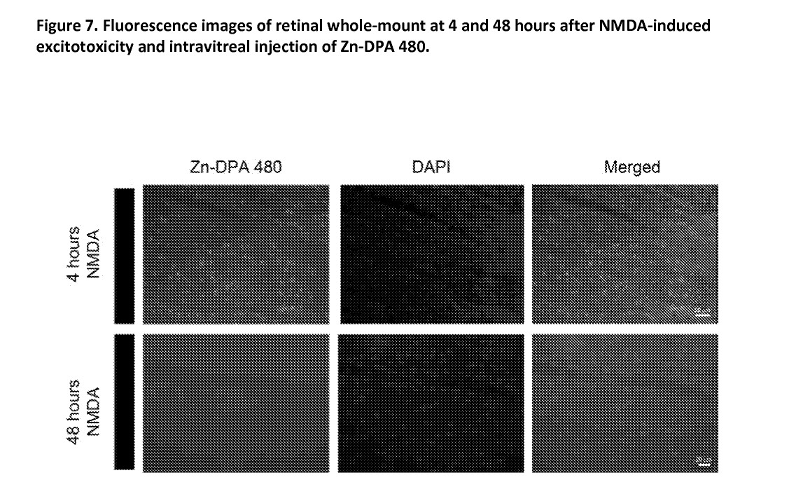

Intravitreal (intraocular) injection of Zn-DPA 480 also labeled retinal neurons after NMDA-induced apoptosis. Intraocular administration of Zn-DPA 480 labeled retinal neurons at 4 hours after NMDA injection but not 48 hours because earlier studies showed that there is no apoptosis in the retina at this time point indicating that the positive labeling of Zn-DPA 480 is a sensitive indicator of membrane changes of apoptotic cells and specific to apoptotic events. Results are illustrated in FIG. 7.

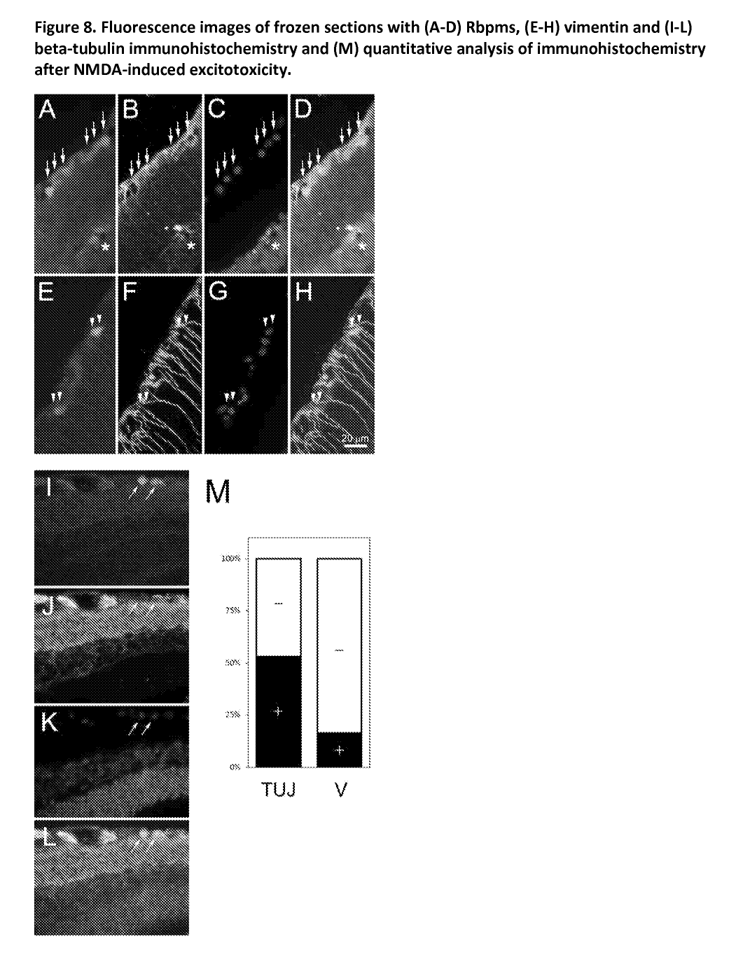

The intravitreal injection of NMDA-induced degeneration specific to retinal ganglion cells, which is known to cause glaucomatous neurodegeneration, and other optic neuropathies. Quantitative analysis showed that majority of Zn-DPA 480 positive cells was retinal ganglion cells but not glial cells after NMDA-induced apoptosis. This finding demonstrates that the labeling of Zn-DPA 480 is specific to the cell type that is dying in the disease. Results are illustrated in FIG. 8.

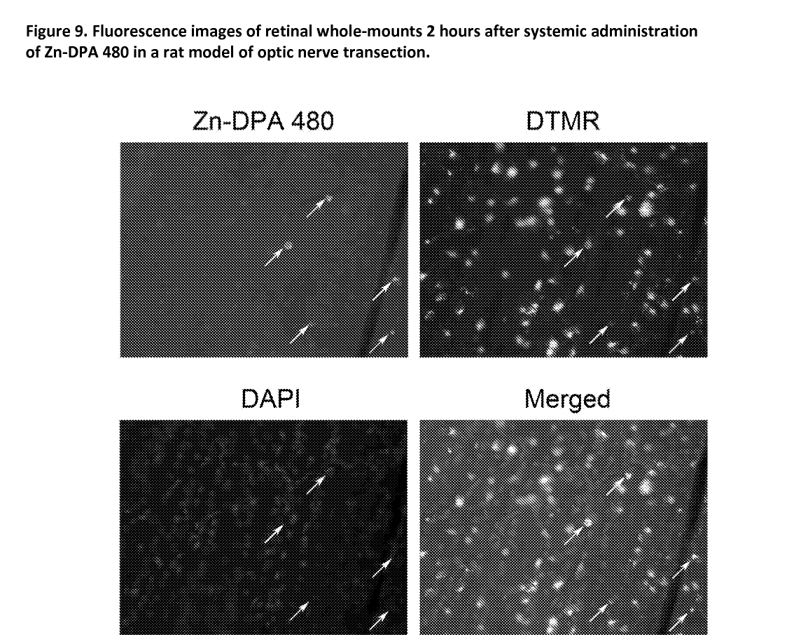

Intravenous (systemic) administration of Zn-DPA 480 labeled dying retinal ganglion cells in the retina after axotomy (transection or surgical cut) of optic nerve in the rats. This finding indicates that Zn-DPA 480 can label apoptotic retinal neurons after trauma, optic neuropathy and other retinal degeneration. Results are illustrated in FIG. 9.

Cell uptake study showed that about 1.5% of 8b (dual functionality) was taken up in paclitaxel-treated U87MG cells after 1 hour of incubation, which is significantly higher than 0.39% observed for 17b (negative control), indicating that the Zn-DPA moiety is indeed the component binding to PS. Results are illustrated in FIG. 10.

Decay-corrected whole-body coronal microPET images of nude mice bearing U87MG tumor (n=5/group) at 1, 2, and 4 hours post-injection of 8b (7.4 MBq) showed significant tumor uptake. Ex vivo PET imaging of tumor and normal tissues of 8b after euthanizing the mice at 4 hours post-injection confirmed the in vivo findings. Results are illustrated in FIG. 11.

The method of the invention may be used to diagnose any applicable ophthalmic conditions and/or diseases, including front of the eye diseases and or back of the eye diseases, which involves the markers that define apoptosis and any related or associated pathways involved in the disease process.

The front of the eye diseases can deal with cellular or subcellular components of the front of the eye anatomy or histology, which includes the acellular tear film layer and its lipid aqueous, mucin components. Upper and lower eyelids including components such as the meibomian gland and its cellular and tissue components including the muscle, lipid producing exo and endocrine glands and its vascular and connective tissue components; conjunctiva and its associated cells including goblet cells, fibroblast cells, vascular and component blood cells; and any conditions or diseases of the corneal layers including the multi layers of epithelial cells, stromal cells and fibroblasts, corneal endothelial cells, corneal nerve and associated cells and ground substances. Diseases of the front of the eye may include: diffuse lamellar keratitis, corneal diseases or opacifications with an exudative or inflammatory component; any disease of the eye that is related to systemic autoimmune diseases; any ocular surface disorders from dry eye, lid margin diseases, meibomian gland disease or dysfunction, dysfunctional tear syndromes, anterior and or posterior blepharitis, Staphlococcal blepharitis, and conjunctival edema anterior uveitis and any inflammatory components or components of the aqueous fluid; inflammatory conditions resulting from surgeries such as LASIK, LASEK, refractive surgery, intraocular lens (IOL) implantation; irreversible corneal edema as a complication of cataract surgery; edema as a result of insult or trauma (e.g., physical, chemical, pharmacological); inflammation; conjunctivitis (e.g., persistent allergic, giant papillary, seasonal intermittent allergic, perennial allergic, toxic, conjunctivitis caused by infection by bacteria, fungi, parasites, viruses or Chlamydia); keratoconjunctivitis (e.g., vernal, atopic, sicca, any ocular surface diseases); any corneal inflammation including infectious keratitis; superficial punctuate keratitis and unspecified keratis; genetic diseases of the cornea (e.g., corneal dystrophies including keratoconus; posterior polymorphous dystrophy; Fuch's dystrophies (corneal and endothelial; aphakic and pseudophakic bullous keratopathy; corneal edema); scleral diseases with or without inflammatory components; and ocular cicatrcial pemphigoid.

The back of the eye diseases can deal with cellular or subcellular components of the back of the eye anatomy and histology including the retina and all of the 10 or more cells comprising the layers of the retina (e.g., inner limiting membrane, retinal ganglion cell layer, inner plexiform layer, inner nuclear layer, outer plexiform layer, photoreceptor layer, outer limiting membrane, inner segment, outer segment, retinal pigment epithelium, Bruck's membrane) and other structures including vitreous and choroid. Additional components of the back of the eye include the ciliary body, iris, uvea and the retinal pigment cells. Back of the eye diseases include processes that involve the optic nerve and all of its cellular and subcellular components such as the axons and their innervations. These include disease such as primary open angle glaucoma, acute and chronic closed angle glaucoma and any other secondary glaucoma. Diseases of the back of the eye also may include myopic retinopathies, macular edema such as clinical macular edema or angiographic cystoid macular edema arising from various etiologies such as diabetes, exudative macular degeneration and macular edema arising from laser treatment of the retina, diabetic retinopathy, age-related macular degeneration, retinopathy of prematurity, retinal ischemia and choroidal neovascularization and like diseases of the retina; genetic disease of the retina and macular (e.g., wet and dry macular degeneration); pars planitis; Posner Schlossman syndrome; Bechet's disease; Vogt-Koyanagi-Harada syndrome; hypersensitivity reactions; toxoplasmosis chorioretinitis; inflammatory pseudo-tumor of the orbit; chemosis; conjunctival venous congestion; periorbital cellulitis; acute dacryocystitis; non-specific vasculitis; sarcoidosis and cytomegalovirus infection.

For detecting fluorescence probes, optical molecular imaging technologies use light emitted through fluorescence and the target reporter molecular can be excited by particular wavelength resulting in the emission of light that can be visualized and recorded by camera such as charge-coupled device camera. This technique can be incorporated into available diagnostic instruments including biomicroscopy (slit lamp), optical coherence tomography, confocal laser scanning microscopy, adaptive optics scanning laserophthalmoloscopy, ophthalmoloscopy and fundus camera.

Multi-modalities are being used to diagnose ocular diseases but not limited to optical imaging, nuclear imaging, and computed tomography. For detecting fluorescence probes, optical molecular imaging technologies use light emitted through fluorescence and the target reporter molecular can be excited by particular wavelength resulting in the emission of light that can be visualized and recorded by camera such as charge-coupled device camera. This technique can be incorporated into available diagnostic instruments including biomicroscope (slit lamp), confocal scanning laser microscope, adaptive optics scanning laser ophthalmoloscope, ophthalmoloscope and fundus camera.

### EXAMPLES

### Example 1

The following example demonstrates labeling of ocular cells by topical administration of Zn-DPA 480 as early as 1 day after corneal toxicity induced by application of benzalkonium chloride (BAC).

Adult Wistar rats were given topical instillation of 2% BAC in 0.9% balanced salt solution (BSS) twice daily. One day after BAC administration, Zn-DPA 480 was topically applied on the corneal surface and animals were euthanized by inhalation of overdose carbon dioxide. The enucleated eyeballs were analyzed by histopathological examination. The fluorescence microscopic examination revealed that scattered corneal cells were positively labeled by Zn-DPA 480 and there was complete loss of the layer of corneal epithelium and decreased cell density of stromal cells. Results are illustrated in FIG. 1.

### Example 2

The following example demonstrates tracking of apoptotic cells by Zn-DPA 480 in corneal toxicity model in the rat.

Adult Wistar rats were given topical instillation of 0.2% BAC in 0.9% balanced salt solution twice daily for 3 days. Then, Zn-DPA 480 was topically applied on the corneal surface and animals were euthanized by inhalation of overdose carbon dioxide. The enucleated eyeballs were processed and analyzed by histopathological examination. The frozen sections were labeled by in situ terminal deoxynucleotidyl transferase-mediated biotin-dUTP nick end labeling (TUNEL). The fluorescence microscopic examination revealed that there were complete loss of the layer of corneal epithelium and increased density of infiltrated cells (polynuclear cells as morphologically shown by DAPI). Increased numbers of TUNEL (red) positive cells and Zn-DPA 480 (green) positive cells were noted in central cornea region and corneal-scleral (limbal) region. Almost all TUNEL positive cells were Zn-DPA 480 positive indicating that Zn-DPA 480 labels dying cells. Results are illustrated in FIG. 1.

### Example 3

The following example demonstrates in vivo imaging of Zn-DPA 480, detection of dying cells and comparison with lissamine green staining in BAC-induced corneal toxicity model in rat.

Adult Wistar rats were given topical instillation of 0.2, 0.1 and 0.02% BAC in 0.9% balanced salt solution (BSS) twice daily for 7 days. External ocular examination was performed and lissamine green staining was used to evaluate the corneal damage prior to and after experiments. One hour after topical Zn-DPA 480 administration, convention light and fluorescence images were taken on animals.

Irregular light reflection and positive lissamine green staining was noted on the cornea after 0.1% and 0.2% BAC administration indicating the presence of corneal surface abnormalities. Punctate Zn-DPA 480 labeling was observed using fluorescence imaging after as low as 0.02% and 0.1% BAC administration. A pooling of Zn-DPA 480 labeling was noted in the central cornea after 0.2% BAC application. There was no remarkable change on the cornea after administration of BSS. Results are illustrated in FIG. 3.

Animals were then euthanized by the inhalation of carbon dioxide and ocular tissues were collected and processed for histopathological examination. There was no TUNEL and Zn-DPA 480 labeling in the cornea after 7 days of BSS (vehicle) administration. No remarkable histological change was noted. After 0.02% BAC administration, a few TUNEL positive cells were observed in the epithelium layer and some were co-localized with Zn-DPA 480 labeling. After 0.1% BAC, there was loss of epithelium layer and increased corneal thickness indicating severe corneal damage and edematous cornea. Compared to the cornea exposed to 0.02% BAC, there were more TUNEL and Zn-DPA 480 positive cells in the superficial corneal layer. After 0.2% BAC, there was an intense labeling of Zn-DPA 480 (green) on the superficial layer and co-localization with TUNEL positive cells. This labeling was corresponding to the pooling of Zn-DPA 480 as shown in FIG. 3. Results are illustrated in FIG. 4.

### Example 4

The following example demonstrates the use of systemic administration of Zn-DPA 480 and Zn-DPA Rhodamine to label retinal ganglion cells in excitoxicity-induced apoptosis model in rats.

Adult Wistar rats were surgically implanted with a retrograde labeling dye, fluoro-gold 2 days before experiment. This procedure was used to pre-label retinal ganglion cells in the retina. To induce retinal ganglion cell degeneration, animals were given intravitreal injection of 3 uL 40 mM N-methyl-D-aspartate (NMDA) in 0.1M PBS. Twenty-four hours after injection, Zn-DPA-Rhodamine or Zn-DPA 480 was intravenously injected. One hour after Zn-DPA administration, the animals were euthanized and the enucleated eyeballs were processed for imaging.

There was double labeling of Zn-DPA-Rhodamine or Zn-DPA 480 and Fluoro-gold after NMDA-induced excitotoxicity indicating Zn-DPA positive cells labeled retinal ganglion cells. Previous studies clearly demonstrated that retinal ganglion cells are the neurons dying through apoptosis in this rat retinal injury model. (Lam, et al. 1999 Invest Ophthalmol Vis Sci. 40:2391-2397; Kwong, et al. 2003 Brain Res. 970(1-2):119-130.) Results are illustrated in FIG. 5.

### Example 5

The following example demonstrates detection of early cell death process in retinal neurons by systemic Zn-DPA administration after NMDA-induced excitoxicity.

Adult Wistar rats were given intravitreal injection of NMDA solution. Animal were euthanized at 2 or 6 hours after NMDA injection (3 uL 40 mM). Two hours prior to euthanasia, Zn-DPA 480 or Zn-DPA Rhodamine was administered intravenously. There were positive Zn-DPA 480 and Zn-DPA-Rhodamine cells in the whole mount retinas at both 2 and 6 hours post-injection. This finding illustrates that both Zn-DPA-Rhodamine and Zn-DPA 480 label retinal cell as early as 2 and 6 hours after NMDA-induced excitotoxicity. Earlier work showed that the peak of TUNEL and DNA fragmentation (a key step of apoptosis) demonstrated by DNA gel electrophoresis is 18 to 24 hours after NMDA injection in rats. (Lam, et al. 1999 Invest Ophthalmol Vis Sci. 40:2391-2397). Results are illustrated in FIG. 6.

### Example 6

The following example demonstrates the tracking of retinal neurons by intraocular administration of Zn-DPA in NMDA-induced excitotoxicity model.

Adult Wistar rats were given intravitreal injection of NMDA solution (3 uL 40 mM). Animals were euthanized 4 and 48 hours after NMDA injection. Solution of Zn-DPA 480 was injected intravitreally 1 hour prior to euthanasia. The retinal tissues were dissected and collected. Fluorescence microscopic examination found that there were many Zn-DPA 480 positive retinal cells at 4 hours post-injection. Absence of Zn-DPA 480 positive cells was noted at 48 hours after NMDA injection. This finding is in concordance with the previous study showing that there is no TUNEL positive cell at 48 hours after NMDA injection. (Lam, et al. 1999 Invest Ophthalmol Vis Sci. 40:2391-2397.) Results are illustrated in FIG. 7.

### Example 7

The following example demonstrates the specificity of labeling retinal neurons in NMDA-induced excitotoxicity model in rats.

Adult Wistar rats were given intravitreal injection of NMDA (3 uL 40 mM). Solution of Zn-DPA 480 was injected intravitreally 1 hour prior to euthanasia. Animals were euthanized at 24 hours post-injection by inhalation of carbon dioxide and the enucleated eyeballs were processed. Frozen sections were collected and immunohistochemistry using antibodies against RNA binding protein with multiple splicing (Rbpms; retinal ganglion cell marker), vimentin (glial cell marker) and beta-tubulin (retinal ganglion cell marker). To evaluate the specificity of Zn-DPA labeling in retinal neurons, the number of Zn-DPA 480 positive cells per frozen section was counted and the percentage of double labeling of Zn-DPA 480 with vimentin and beta-tubulin was determined. Use of antibody against Rbpms was valid to quantify the survival of retinal ganglion cell in various optic neuropathies in the rats (Kwong, et al. 2010. Invest Ophthalmol Vis Sci; 51:1052-1058; Kwong, et al. 2011. Invest Ophthalmol Vis Sci; 52:9694-9702.). Imaging of whole mount retina showed that Zn-DPA 480 positive cells were co-localized with Rbpms immunoreactivity (FIG. 9A-9D). Microscopic examination demonstrated that Zn-DPA 480 positive cells were not labeled by vimentin (FIG. 9E-9H) but labeled by beta-tubulin (FIG. 9I-9L). The quantitative analysis showed that approximately 53% and 27% of Zn-DPA 480 were beta-tubulin and vimentin positive respectively. Since around 50% of cells in the layer of retinal ganglion cells in rodent retina are displaced amacrine cells, the finding supports that Zn-DPA 480 mainly labeled retinal ganglion cells. Results are illustrated in FIG. 8.

### Example 8

The following example demonstrates the tracking of dying neurons by Zn-DPA in axotomy-induced optic neuropathy in the rat.

Adult Wistar rats were used in this experiment. Optic nerve was surgically exposed and transected to cause optic neuropathy with retinal ganglion cell degeneration. Retrograde labeling dye, DTMR, was implanted to pre-label retinal ganglion cells. Animals were euthanized and ocular tissues were evaluated at 1 week after optic nerve axotomy. Two hours prior to euthanasia, solution of Zn-DPA 480 was administered intravenously. The retina were processed and examined under fluorescence microscope. There were co-localization of Zn-DPA 480 and DTMR (arrows) in the retina. All Zn-DPA 480 cells were DTMR positive indicating that Zn-DPA 480 labels retinal ganglion cells after axotomy-induced optic nerve neuropathy. Results are illustrated in FIG. 9.

### Example 9

### Example 9-1

### Exemplary Reaction Schemes for Forming Dual Modality Radiolabeled and Fluorescent Dipicolylamine Derivatives, Part One

This Example illustrates methods that may be used in various embodiments for the synthesis of non-radioactive and radioactive fluorine-containing fluorescent compounds (8a) and (8b). In various embodiments, compounds in accordance with the present disclosure may be synthesized according to Scheme 1.

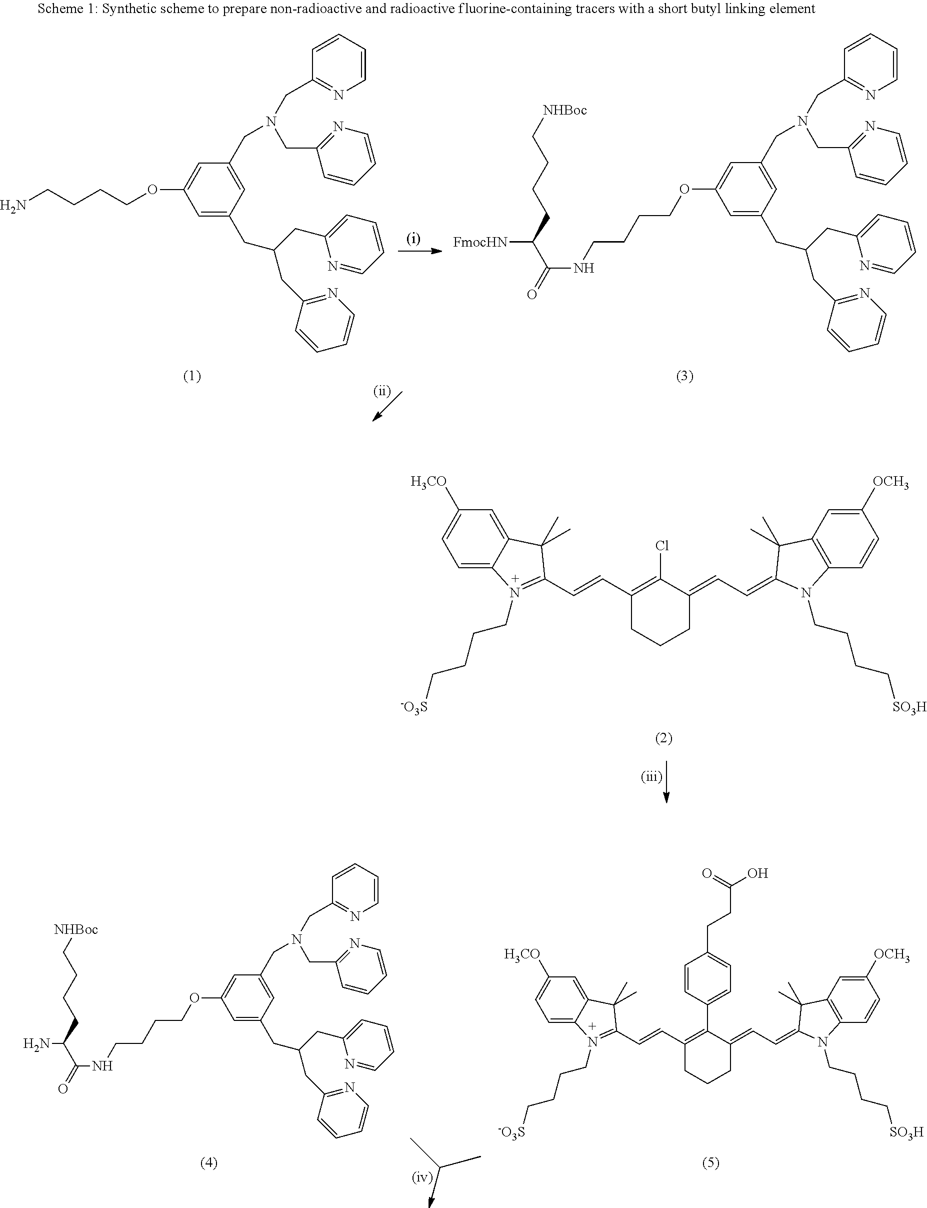

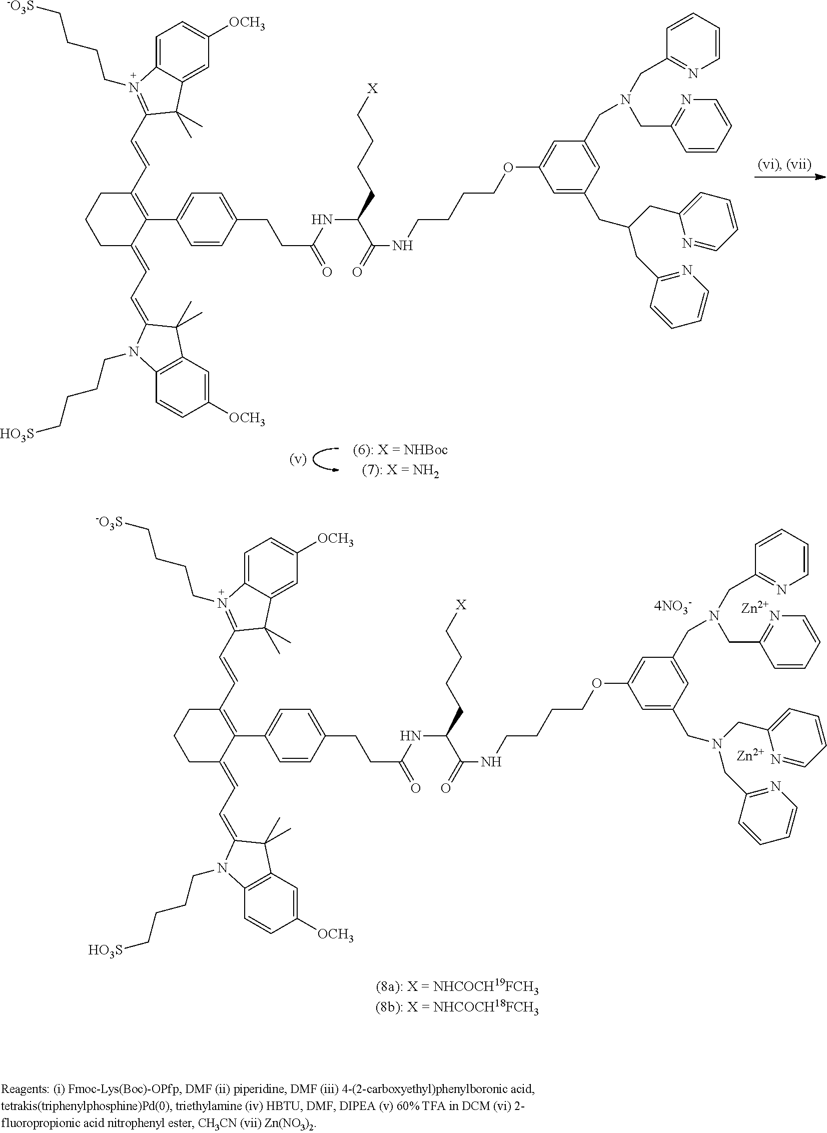

In various embodiments, compounds (1) and (2) may be prepared via procedures known to those of skill in the art (see, e.g., Lakshmi et al., (2004) Tetrahedron, 60, 11307-11315; Narayanan & Patonay (1995) J. Org. Chem. 60, 2391-2395).

Synthesis of (3):

A solution of (1) (215 mg, 0.366 mmol) and Fmoc-Lys(Boc)-OPfp (260 mg, 0.410 mmol, BAChem) in 3 mL of DMF was stirred overnight at room temperature. The solution was then concentrated and purified by silica gel column chromatography eluting with 4% ammonium hydroxide in CH₃CN and then 5% ammonium hydroxide in CH₃CN to provide 281 mg of (3) as a pale yellow oil. ¹H NMR (CDCl₃, 400 MHz): δ 8.51 (d, 4H, J=4.6 Hz), 7.75 (d, 2H, J=7.5 Hz), 7.65-7.54 (m, 9H), 7.38 (t, 2H, J=7.5 Hz), 7.30 (d, 1H, J=8.4 Hz), 7.18-7.11 (m, 4H), 7.07 (s, 1H), 6.83 (s, 2H), 6.34-6.24 (m, 1H), 5.59-5.50 (m, 1H), 4.73-4.60 (m, 1H), 4.47-4.36 (m, 2H), 4.20 (t, 1H, J=6.8 Hz), 4.13-4.05 (m, 1H), 3.95 (t, 2H, J=5.8 Hz), 3.80 (s, 8H), 3.64 (s, 4H), 3.39-3.24 (m, 2H), 3.16-3.00 (m, 2H), 1.93-1.78 (m, 2H), 1.75-1.59 (m, 2H), 1.54-1.32 (m, 9H).

Synthesis of (4):

Treatment of (3) with a solution of piperidine in DMF (1:3) overnight followed by solvent concentration furnished a crude material. Purification was carried out by silica gel chromatography using an increasing concentration of ammonium hydroxide (2-10%) in CH₃CN provided 217 mg of pure (4). ¹H NMR (CDCl₃, 400 MHz): δ 8.51 (d, 4H, J=4.9 Hz), 7.68-7.55 (m, 8H), 7.17-7.10 (m, 4H), 7.06 (s, 1H), 6.84 (s, 2H), 4.68-4.58 (m, 1H), 3.97 (t, 2H, J=6.0 Hz), 3.80 (s, 8H), 3.65 (s, 4H), 3.40-3.28 (m, 3H), 3.16-3.06 (m, 2H), 1.92-1.79 (m, 3H), 1.76-1.66 (m, 2H), 1.57-1.47 (m, 3H), 1.46-1.42 (m, 9H).

Synthesis of (5):

To a solution of (2) (0.50 g, 0.636 mmol) in methanol (10 mL) and water (2.5 mL) was added tetrakis(triphenylphosphine)Pd(0) (89 mg, 0.775 mmol) and 4-(2-carboxyethyl)phenylboronic acid (0.27 g, 1.41 mmol) followed by triethylamine (1.25 mL). The resulting solution was stirred at room temperature overnight and then purified by silica gel chromatography eluting with an increasing amount of methanol in dichloromethane (5% to 30%) to provide pure (5; 400 mg, 64%). ¹H NMR (DMSO-d₆, 400 MHz): δ 7.47 (d, 2H, J=7.9 Hz), 7.28 (d, 2H, J=8.9 Hz), 7.18-7.10 (m, 4H), 7.07 (d, 2H, J=14.0 Hz), 6.90 (dd, 2H, J=2.3, 6.4 Hz), 6.14 (d, 2H, J=14.1 Hz), 4.21-3.99 (m, 6H), 3.76 (s, 6H), 2.71-2.58 (m, 6H), 2.46 (t, 4H, J=7.2 Hz), 2.00-1.86 (m, 2H), 1.80-1.59 (m, 8H), 1.10 (s, 12H). ESI-MS m/z C₄₉H₅₉N₂O₁₀S₂ calcd, 900.37. found, 901.50. [M+H]⁺.

Synthesis of (6):

To a solution of (4) (54 mg, 0.066 mmol) in DMF (2 mL) was added a solution of (5) (60 mg, 0.667 mmol) in anhydrous DMSO then solid HBTU (32 mg, 0.084 mmol) and a few drops of DIPEA. The resulting solution was stirred at room temperature for 1 hour. The reaction mixture was then purified by silica gel chromatography eluting with 20% methanol in dichloromethane and then gradually increasing the methanol to 25% and ammonium hydroxide from 0 to 3% to provide 75 mg of (6). ¹H NMR (DMSO-d₆, 400 MHz): δ 8.48 (d, 4H, J=4.0 Hz), 7.78-7.70 (m, 4H), 7.57 (d, 4H, J=7.8 Hz), 7.44 (d, 2H, J=8.0 Hz), 7.31-7.20 (m, 6H), 7.15-7.10 (m, 4H), 7.08 (s, 2H), 7.04 (s, 1H), 6.89 (dd, 2H, J=6.4, 2.4 Hz), 6.80 (s, 2H), 6.13 (d, 2H, J=14.2 Hz), 4.09-4.01 (m, 4H), 3.93 (t, 2H, J=6.3 Hz), 3.74 (s, 6H), 3.70 (s, 8H), 3.58 (s, 4H), 3.17 (d, 2H, J=5.2 Hz), 3.01-2.93 (m, 2H), 2.91-2.83 (m, 2H), 2.69-2.61 (m, 4H), 2.46 (t, 4H, J=7.2 Hz), 1.98-1.87 (m, 2H), 1.78-1.60 (m, 8H), 1.60-1.48 (m, 2H), 1.34 (s, 12H), 1.30-1.18 (m, 10H), 1.11-1.01 (m, 9H).

Synthesis of Compound (7):

Compound (6) (70 mg) was stirred in a solution of TFA:CH₂Cl₂ (60:40) at room temperature for 6 hours, then concentrated to provide (7). ESI-MS m/z C₉₄H₁₁₄N₁₁O₁₂S₂ calcd, 1,597.79. found, 1,598.75. [M+H]⁺, 800.24 [M+2H]²⁺.

Synthesis of (8a):

2-fluoropropionic acid (9.6 mg, 0.104 mmol), disuccinimidyl carbonate (100 mg, 0.390 mmol), and triethylamine (55 μL) were stirred together in anhydrous DMF (1 mL) for 22 hours. Compound (7) (50 mg, 0.0313 mmol) in DMF (0.5 mL) containing triethylamine (50 uL) was then added and the mixture stirred for 45 minutes. The reaction mixture was then concentrated and purified via reverse-phase HPLC to provide apo-(8a). ESI-MS m/z C₉₄H₁₁₄FN₁₁O₁₂S₂ calcd, 1,671.81. found, 1,672.76. [M+H]⁺, 837.28 [M+2H]²⁺. Treatment of apo-(8a) with 2.1 equivalents of zinc nitrate in methanol followed by removal of solvent provides (8a).

### Example 9-2

### Exemplary Reaction Schemes for Forming Radiolabeled Dipicolylamine Derivatives, Part Two

This Example illustrates methods for a synthetic scheme that may be used in various embodiments to prepare non-radioactive and radioactive fluorine containing fluorescent tracers with polyethylene glycol (PEG) and/or amino acid (AA) linking elements. In various embodiments, the incorporation of different linkers may affect the overall hydrophilic/hydrophobic balance of small molecules. Thus, in various embodiments, achieving the correct hydrophilic/hydrophobic balance may be critical to obtaining favorable pharmacokinetics and target/background (T/B) ratios. In addition to tracer (8b), which has a short lipophilic butyl linker, other types of pharmacokinetic modifying groups, such as polyethylene glycol units or amino acid sequences may be incorporated into the dual modality tracer. For example, in various embodiments, the PEG₄ linker may increase tracer hydrophilicity while preserving overall charge, and may improve tumor uptake and excretion kinetics of various small peptide receptor-targeted radiopharmaceuticals. As another example, the Asp₂ amino acid linker may enhance hydrophilicity and also modify charge (e.g., introduce two negative carboxyl groups), and this type of modification may reduce accumulation of renal radioactivity.

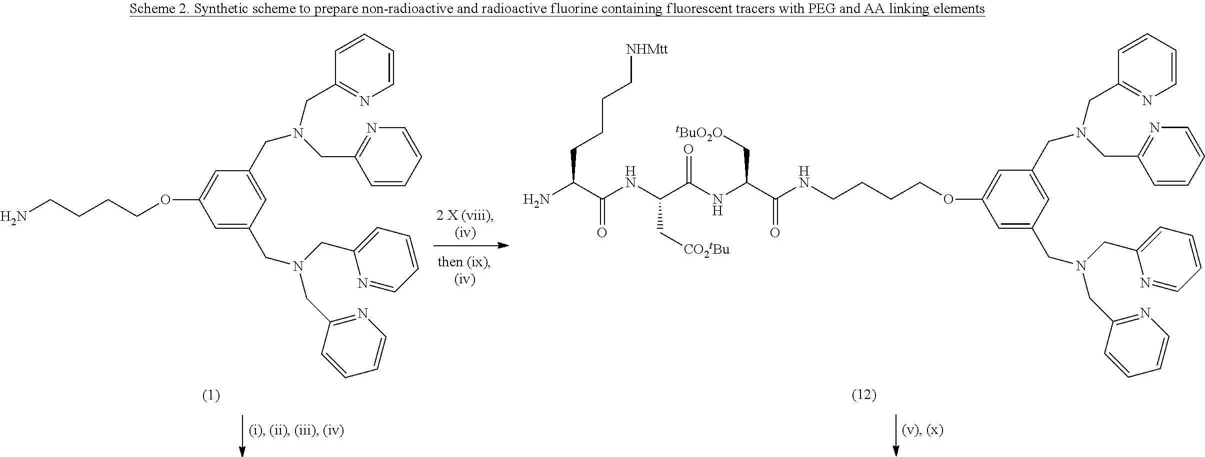

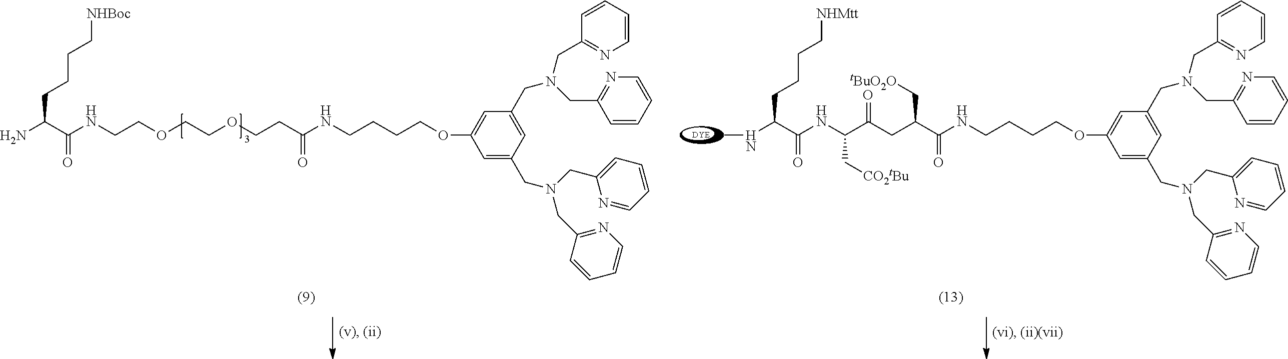

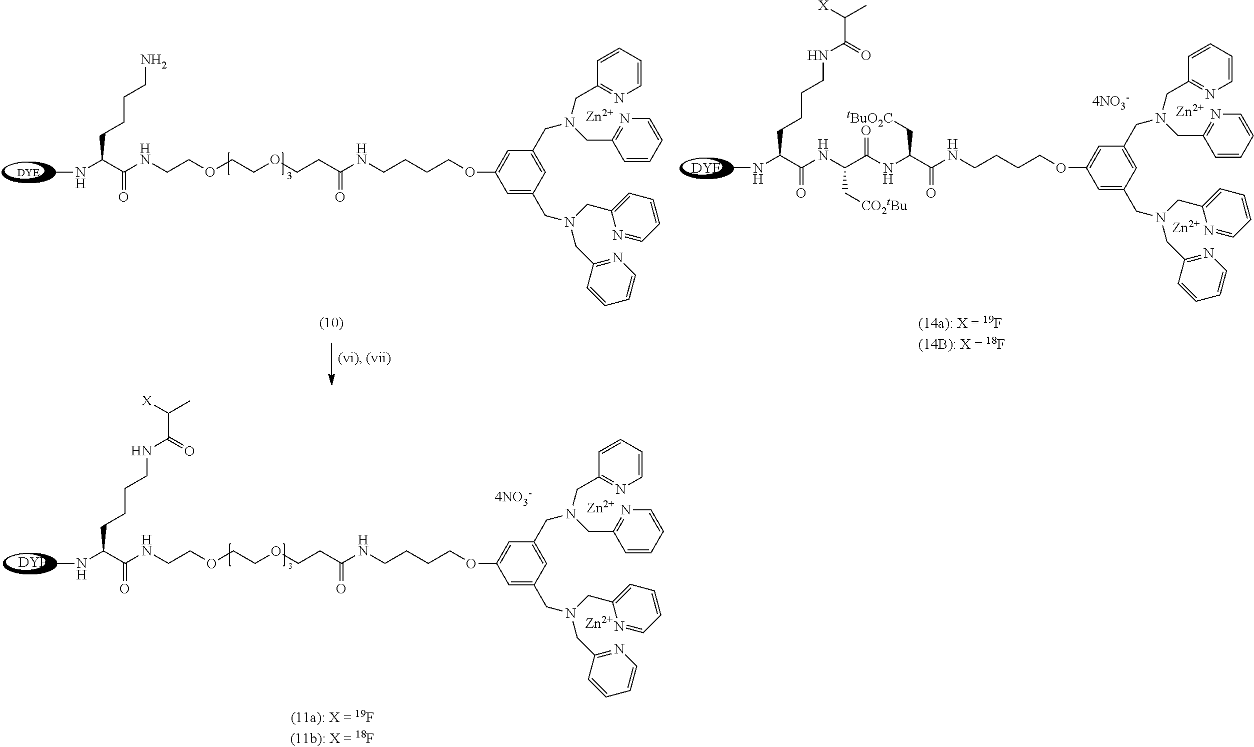

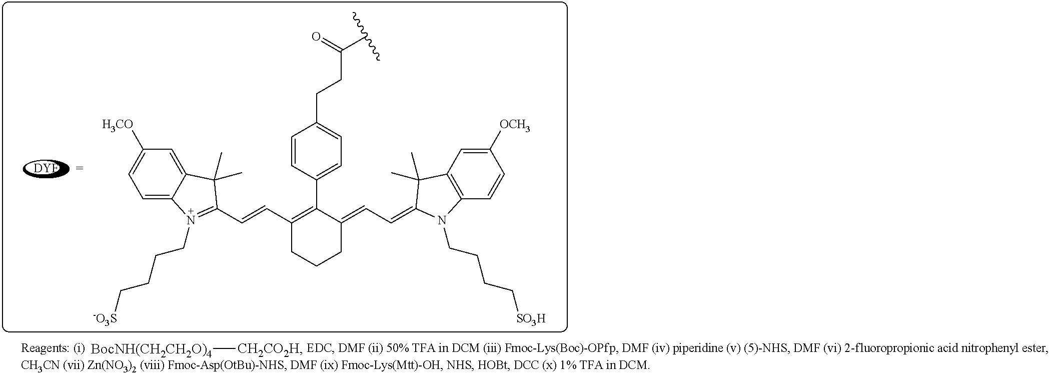

In various embodiments, the ¹⁹F reference compounds (11a) and (14a) may be prepared as shown in Scheme 2. Compound (1) was coupled with the PEG₄ derivative, BocNH(CH₂CH₂O)₄CH₂CH₂CO₂H (Quanta Biodesign, Inc.) under standard peptide coupling conditions. Removal of the Boc protecting group with TFA followed by coupling with the lysine analog, Fmoc-Lys(Boc)-OPfp, and subsequent removal of the Fmoc protecting group with piperidine may then furnish (9). Carboxylic acid dye (5) may be coupled to (9) under standard conditions, followed by the Boc group removal to provide (10). Reaction of (10) with [¹⁹F]NFP [45] followed by chromatographic purification and addition of 2.1 equivalents of zinc nitrate may provide (11a). Preparation of (14a) initially involves 3 cycles of standard amino acid coupling/deprotection reactions with (1), using Fmoc-Asp(OtBu)-NHS (2 cycles), then Fmoc-Lys(Mtt)-NHS to provide (12). Coupling of (12) with carboxylic acid dye (5) followed by selective removal of the Mtt protecting group may provide amine (13). The 2-fluoropropionyl group may be incorporated as before, then the t-Butyl groups may be quickly removed (TFA, 30 minutes, room temperature) and zinc added to provide (14a).

### Example 9-3

### Reaction Schemes for Forming Radiolabeled Dipicolylamine Derivatives, Part Three

This Example illustrates synthetic schemes that may be used to form analogs of non-radioactive and radiolabeled dipicolylamine derivatives in various embodiments.

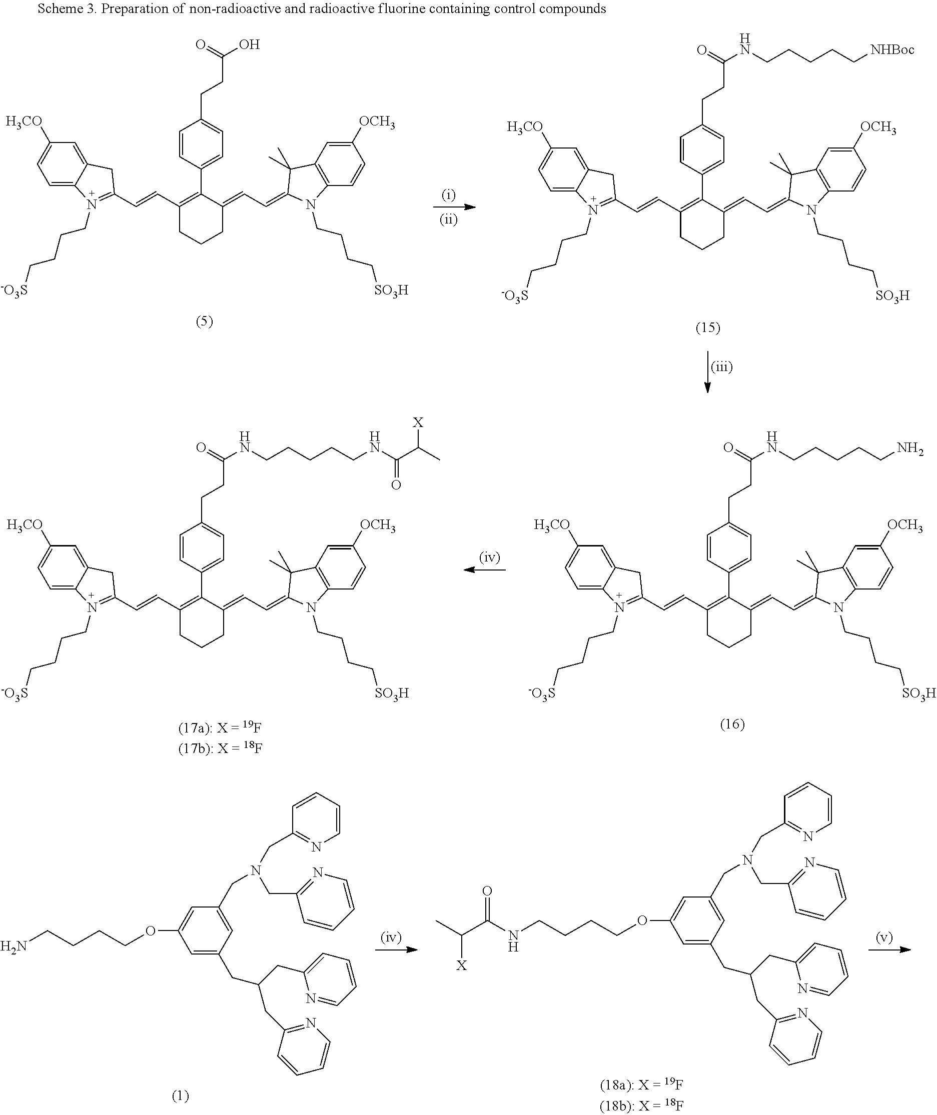

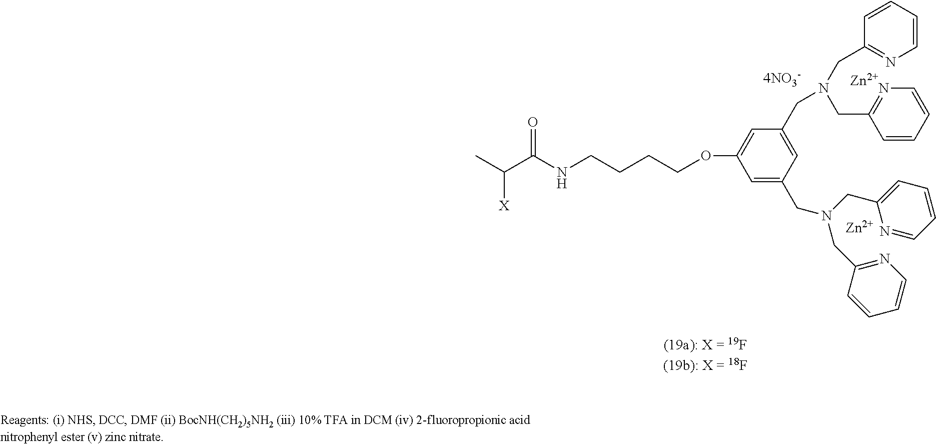

### Synthesis of Radioactive ¹⁸F-Labeled Fluorescent Tracers (8b), (11b) and (16b):

The radiosynthesis of [¹⁸F]NFP was performed as previously described (see, e.g., Chin et al., (2012) Mol. Imaging Biol. 14, 88-95). About 1 mg of (7) [or (10); or (13)] in 0.1 mL of DMSO containing 20 μL of diisopropylethylamine was added to the [¹⁸F]NFP-containing vial and heated at 80° C. for 10 minutes. The mixture was then cooled and diluted with 0.7 mL of water containing 25 μL of acetic acid and loaded onto a semi-prep HPLC column [in the case of (13) a TFA treatment was added to remove t-Butyl groups]. The desired product was collected, concentrated, and incubated with 100 μL of 4.2 mM zinc nitrate at 40° C. for 10 minutes. The final product was passed through a 0.22-μm Millipore filter into a sterile dose vial for use.

In various embodiments, the ¹⁹F reference compounds (17a) and (19a) may be prepared as shown in Scheme 3.

### Synthesis of Non-Radioactive ¹⁹F-Control Compounds (17a) and (19a)

Preparation of (15):

To a solution of dye (5) (100 mg, 0.111 mmol) in 2 mL of DMF was added N-hydroxysuccinimide (76 mg, 0.666 mmol) followed by N,N′-dicyclohexylcarbodiimide (134 mg, 0.666 mmol) in 2 mL of DMF. The solution was stirred at room temperature overnight. N-Boc-cadaverine (70 μL) in 1 mL of DMF was then added and the mixture stirred for 1 hour and concentrated. The product was purified by silica gel chromatography eluting with increasing amounts of methanol (5% to 35%) in dichloromethane to yield 90 mg of (15). ¹H NMR (DMSO-d₆, 400 MHz): δ 7.44 (d, 2H, J=7.7 Hz), 7.28 (d, 2H, J=8.8 Hz), 7.18-7.02 (m, 6H), 6.90 (dd, 2H, J=6.6, 2.0 Hz), 6.14 (d, 2H, J=14.1 Hz), 4.14-4.00 (m, 4H), 3.76 (s, 6H), 3.14-3.04 (m, 2H), 3.02-2.93 (m, 2H), 2.93-2.84 (m, 2H), 2.71-2.61 (m, 4H), 2.48-2.42 (m, 6H), 1.98-1.87 (m, 2H), 1.79-1.62 (m, 10H), 1.37 (s, 9H), 1.30-1.20 (m, 4H), 1.11 (s, 12H).

Preparation of (16):

Compound (15) (90 mg, 0.083 mmol) was dissolved in 5 mL of solution (10% TFA:90% CH₂Cl₂) and stirred at room temperature for 2 hours. The reaction mixture was then concentrated and placed in a vacuum oven at 50° C. for a few hours to dry to provide (16). ESI-MS m/z C₅₄H₇₂N₄O₉S₂ calcd, 984.47. found, 985.63. [M+H]⁺, 493.27 [M+2H]²′.

Preparation of (17a):

To a solution of 2-fluoropropionic acid (7.8 mg, 84.5 μmol) in 0.5 mL anhydrous acetonitrile was added TSTU (17.6 mg, 58.5 μmol). The pH of the solution was adjusted to 8.5-9.0 by DIPEA. The reaction mixture was stirred at room temperature for 0.5 hours, and then compound (16) (3 μmol) in DMF was added in one aliquot. After being stirred at room temperature for 2 hours, the product was isolated by semi-preparative HPLC to provide (17a). ESI-MS m/z C₅₇H₇₅FN₄O₁₀S₂ calcd, 1,058.49. found, 1059.53. [M+H]⁺.

Preparation of (18a):

To a solution of 2-fluoropropionic acid (7.8 mg, 84.5 μmol) in 0.5 mL anhydrous acetonitrile was added TSTU (17.6 mg, 58.5 μmol). The pH of the solution was adjusted to 8.5-9.0 by DIPEA. The reaction mixture was stirred at room temperature for 0.5 hours, and then compound (1) (3 μmol) in DMF was added in one aliquot. After being stirred at room temperature for 2 hours, the product was isolated by semi-preparative HPLC to provide (18a). ESI-MS m/z C₃₉H₄₄FN₇O₂ calcd, 661.35. found, 662.43. [M+H]⁺, 684.42 [M+Na]⁺.

Preparation of (19a):

(18a) was incubated with 2.1 equivalents of zinc nitrate in water at 40° C. for 10 minutes to provide (19a).

### Synthesis of ¹⁸F-Control Compounds (17b) and (19b)

Preparation of (17b):

About 1 mg of (16) in 0.1 mL of DMSO containing 20 μL of diisopropylethylamine was added to the [¹⁸F]NFP-containing vial and heated at 80° C. for 10 minutes. The mixture was then cooled and diluted with 0.7 mL of water containing 25 μL of acetic acid and loaded onto a semi-prep HPLC column. The desired product was collected, concentrated, and passed through a 0.22-μm Millipore filter into a sterile dose vial for use.

Preparation of (19b):

About 1 mg of (1) in 0.1 mL of DMSO containing 20 μL of diisopropylethylamine was added to the [¹⁸F]NFP-containing vial and heated at 80° C. for 10 minutes. The mixture was then cooled and diluted with 0.7 mL of water containing 25 μL of acetic acid and loaded onto a semi-prep HPLC column. The desired product was collected, concentrated, and incubated with 100 μL of 4.2 mM zinc nitrate at 40° C. for 10 minutes. The final product was passed through a 0.22-μm Millipore filter into a sterile dose vial for use.

### Example 10

Cell uptake study to show the binding of F-18 radiolabeled dual molecule targeted cells with phosphatidyserine exposed: U87MG human glioblastoma cells were seeded into 48-well plates at a density of 1.0×10⁵ cells per well 24 hours prior to the study. U87MG cells were then incubated with 8b (370 kBq/well) at 37° C. for 15, 30, and 60 minutes. After incubation, tumor cells were washed three times with ice cold PBS and harvested by trypsinization with 0.25% trypsin/0.02% EDTA (Invitrogen, Carlsbad, Calif.). At the end of trypsinization, wells were examined under a light microscope to ensure complete detachment of cells. Cell suspensions were collected and measured in a gamma counter (Perkin-Elmer Packard Cobra). Cell uptake data was presented as percentage of total input radioactivity after decay correction. Experiments were performed twice with triplicate wells. After 1 hour of incubation, about 1.5% of 8b was taken up in paclitaxel-treated U87MG cells, which is significantly higher than 0.39% observed for 17b (FIG. 10), indicating that the Zn-DPA moiety is indeed the component binding to PS.

### Example 11

Animal model: This Example illustrates the efficacy of the F-18 labeled Zn-DPA multimodality compounds at detecting phosphatidylserine in an animal model.

Female athymic nude mice (about 4-6 weeks old, with a body weight of 20-25 g) were obtained from Harlan Laboratories (Livermore, Calif.). The U87MG human glioma xenograft model was generated by subcutaneous injection of 5×10⁶ U87MG human glioma cells into the front flank of female athymic nude mice. The tumors were allowed to grow 3-5 weeks until 200-500 mm³ in volume. Tumor (target) growth was followed by caliper measurements of the perpendicular dimensions.

MicroPET scans and imaging analysis were performed using a rodent scanner (microPET R4 scanner; Siemens Medical Solutions). About 7.4 MBq of radiolabeled probe (8b) was intravenously injected into each mouse (n=5) under isoflurane anesthesia. Five-minute static scans were acquired at 1, 2, and 4 hours post-injection. The images were reconstructed by a two-dimensional ordered-subsets expectation maximum (OSEM) algorithm. For each microPET scan, regions of interest were drawn over the tumor, normal tissue, and major organs on the decay-corrected whole-body coronal images. The radioactivity concentration (accumulation) within the tumor, muscle, liver, and kidneys were obtained from the mean value within the multiple regions of interest and then converted to % ID/g. At 4 hours after intravenous injection of radiolabeled probe (8b), mice were sacrificed and dissected. U87MG tumor, major organs, and tissues were collected and scanned using a five-minute static protocol. Representative decay-corrected coronal images at different time points are shown in FIG. 11A. The U87MG tumors were all clearly visible with high contrast to contralateral background at all time points measured beginning 1 hour after injection of 8b. Ex vivo PET imaging of tumor and normal tissues of 8b confirmed the in vivo findings using F-18 labeled dual molecule (FIG. 11B).

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A method for diagnosing an ocular disease characterized by an increase in the extracellular level of phosphatidylserine in a human or living subject, comprising:
(a) administering an effective amount of a single or dual-modality compound comprising a fluorescence moiety with or without a radioactive moiety, a linker, and an anionic surface-targeting moiety; and
(b) measuring the fluorescent emission of the fluorescent compound to obtain an image, thereby determining the site of an ocular disease,
(c) measuring the radioactive emission of the radioactive moiety if present to obtain an image, thereby determining the site of an ocular disease,
(d) measuring both the fluorescent emission of the fluorescent compound and radioactive emission of the radioactive moiety if present to obtain an image, thereby determining the site of an ocular disease,
wherein the anion surface-targeting moiety comprises an organometallic complex.

2. The method of claim 1, wherein the ocular disease is a front of the eye disease.

3. The method of claim 1, wherein the ocular disease is a back of the eye disease.

4. The method of claim 1, wherein the organometallic complex comprises a metal cation complexed to one or more aryl ligands.

5. The method of claim 4, where the metal cation is a zinc or copper cation.

6. The method of claim 1, wherein the fluorescent moiety has an emission max range of 440 nm-900 nm and an absorbance max range of 380 nm-880 nm.

7. The method of claim 1, wherein the fluorescent moiety is a fluorescein, a rhodamine, BODIPY, Cy-3, Cy-5, Cy-7, a squaring rotaxane, an NIR dye, or a derivative thereof.

8. The method of claim 1, wherein the radioactive entity is a radionuclide.

9. The method of claim 8, wherein the radionuclide includes, but is not limited to, ¹⁸F, ⁶⁴Cu, ⁶⁸Ga, ^{99m}Tc, ¹¹¹In, ¹²³I, ¹²⁴I, ⁹⁰Y, ¹⁷⁷Lu, ¹¹C, ¹⁴C, ³H, ³²P, ³³P, ¹⁸⁶Re, ¹⁸⁸Re, or ⁸⁶Zr.

10. The method of claim 1, wherein the linker comprises a heterocycle.

11. The method of claim 1, wherein the linker comprises a hydrophilic moiety selected from the group consisting of: hydroxyl, carbonyl, sulfonamide, sulfonate, phosphate, aliphatic hydrocarbon, polyethylene glycol moiety, polar amino acid moiety, peptide, sugar mimetic, and sugar moiety.

12. The method of claim 10, wherein the linker is selected from the group consisting of: 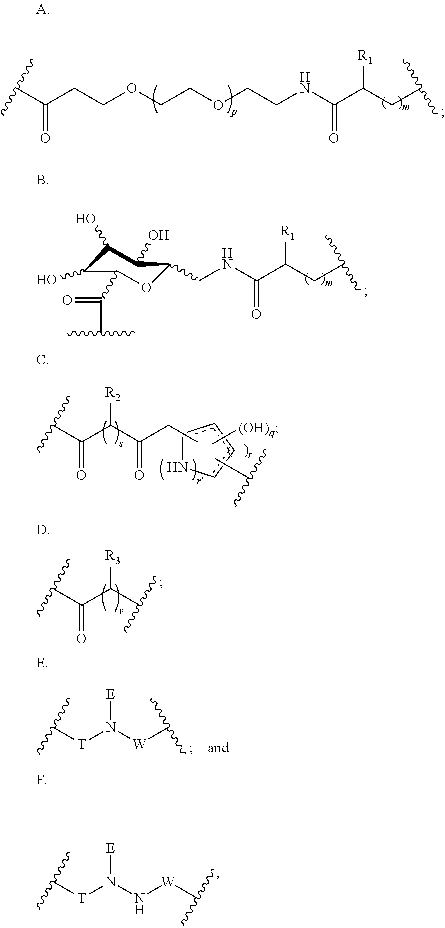
wherein E is: 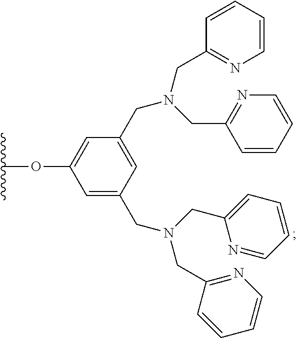
wherein T is selected from the group consisting of: 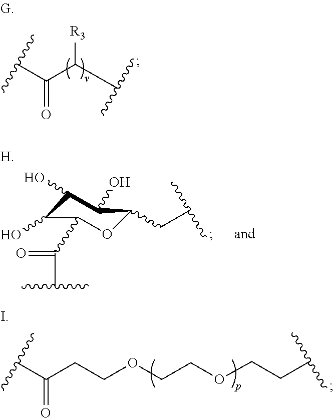
wherein W is selected from the group consisting of: 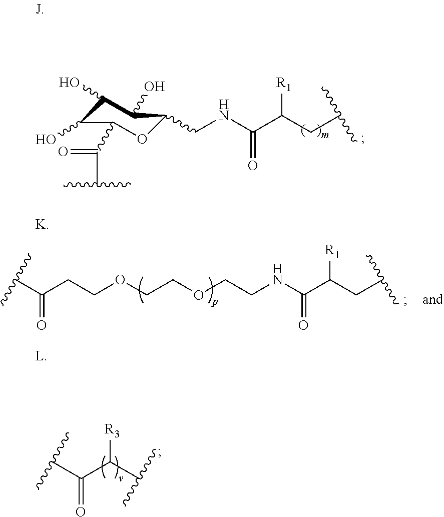
and
wherein
each R₁ is selected from the groups consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, and C₁-C₆-alkoxy-C₁-C₆-alkyl, wherein the alkyl, alkenyl, alkynyl, alkyloxy, aryl, carbocycle, heterocycle, and substitutions thereof;
each R₂ is independently selected from the group consisting of —H, —OH, C₁-C₆ alkyl, C₂-C₆ alkenyl, C₂-C₆ alkynyl, C₁-C₆ alkyloxy, aryl-(C₁-C₆ alkylene)-, hydroxy-C₁-C₆-alkyl, and C₁-C₆-alkoxy-C₁-C₆-alkyl; wherein the alkyl, alkenyl, alkynyl, alkyloxy, and aryl-alkylene groups are each optionally substituted;
each R₃ is independently selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, C₁-C₆-alkoxy-C₁-C₆-alkyl, and a PEG moiety; wherein the alkyl, alkenyl, alkynyl, alkoxy, aryl, carbocycle, and heterocycle groups are each optionally substituted;
each v is independently selected from 0, 1, 2, 3, and 4;
m is 0, 1, 2, 3 or 4;
p is an integer between 1 and 110;
q is 1, 2, 3 or 4;
r is 1, 2 or 3;
r′ is 0 or 1; and
s is 1, 2, 3 or 4.

13-15. (canceled)

16. The method of claim 1, where the ocular disease involves apoptotic or necrotic cell death.

17. The method of claim 1, wherein the compound has the structural formula: 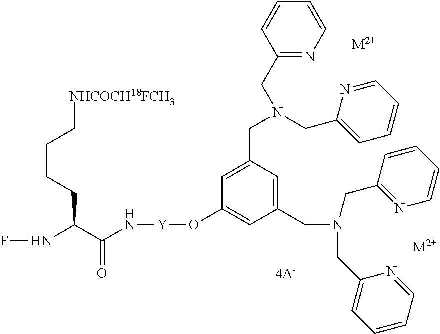
wherein, F is a near infrared fluorescent dye; M is a metal ion; Y is a linker to modulate pharmacokinetics; and A is a pharmaceutically acceptable anion.

18. The method of claim 1, wherein the compound has the structural formula:

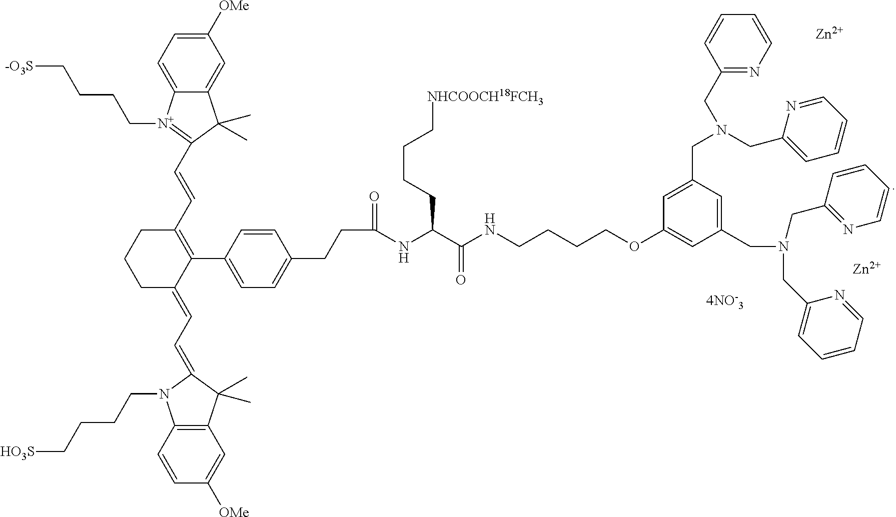

19. The method of claim 1, wherein the compound has the structural formula:

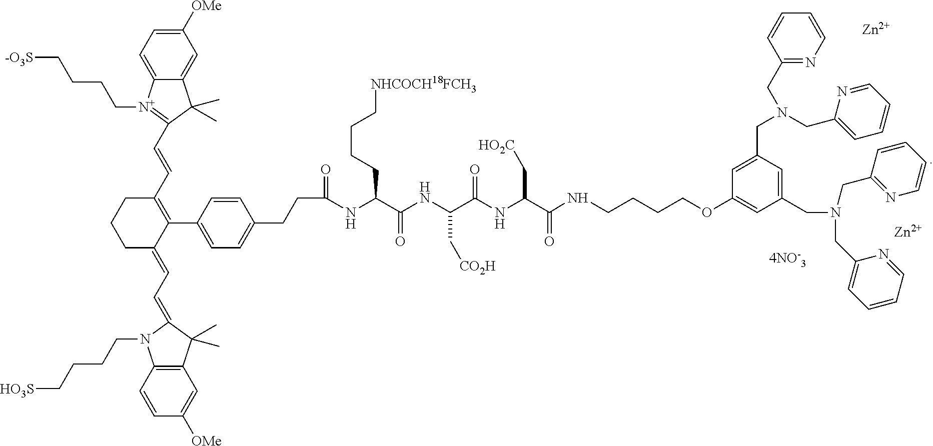

20. The method of claim 1, wherein the compound has the structure:

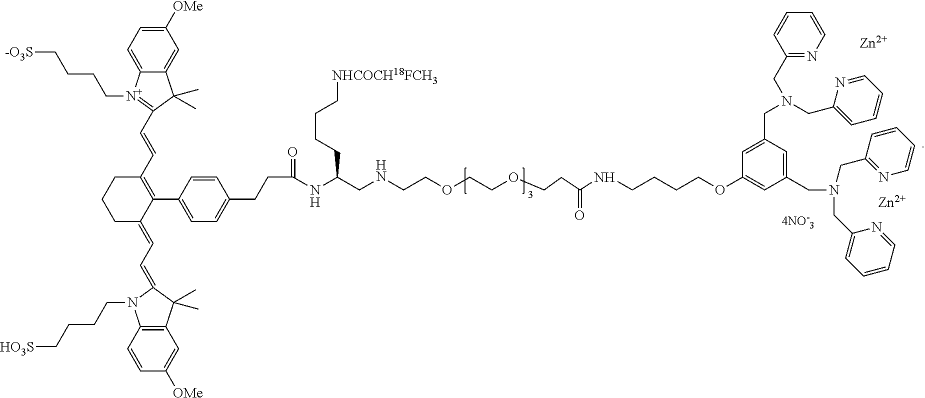

21-23. (canceled)

24. The method of claim 1, wherein the compound has the structure:

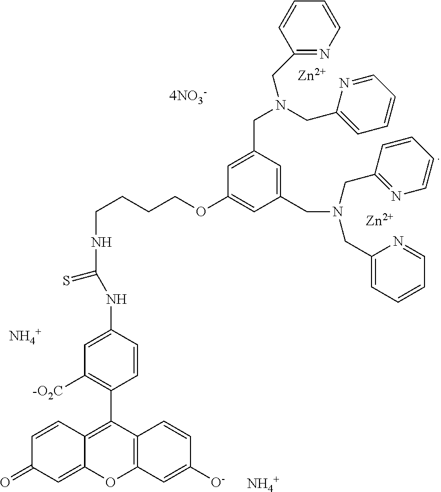

25. The method of claim 1, wherein the compound has the structure:

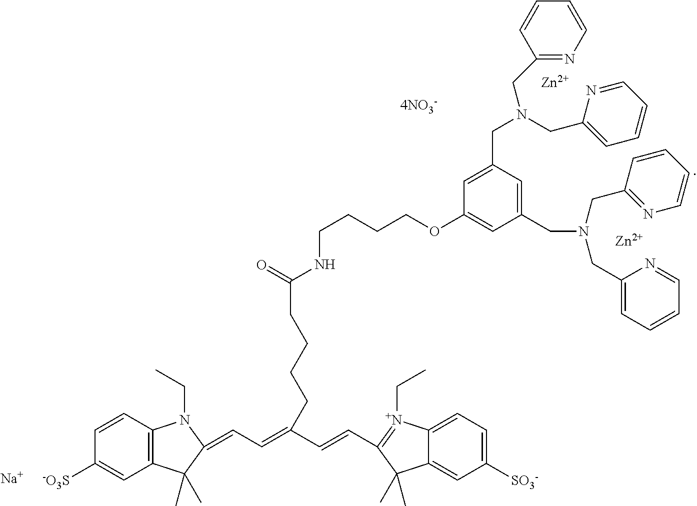

26. The method of claim 1, wherein the compound has the structure:

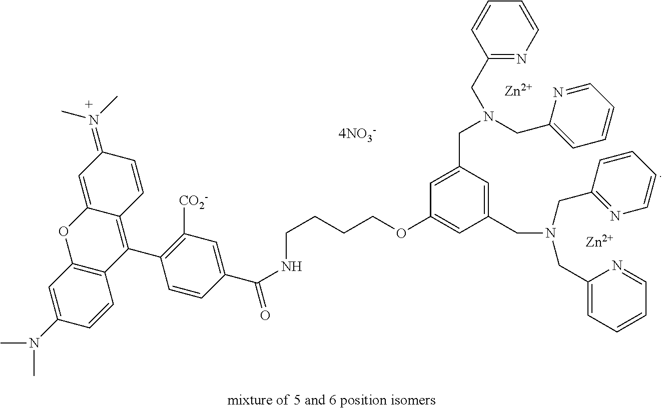

27. The method of claim 1, wherein the compound has the structure:

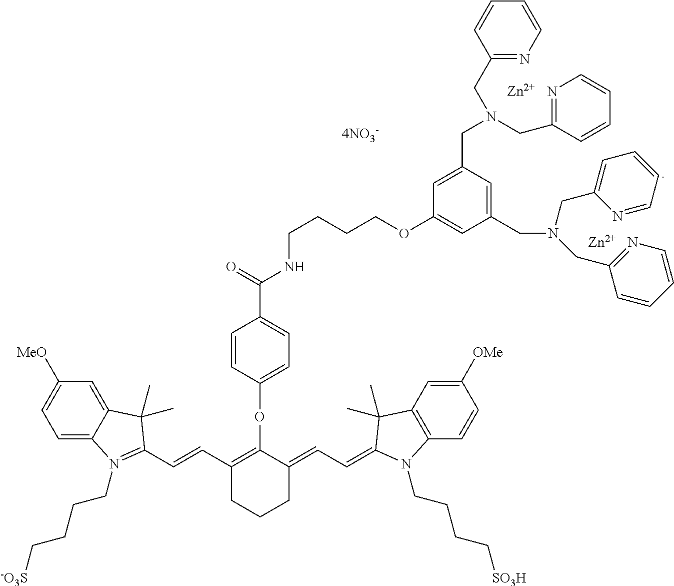

28. The method of claim 1, further comprising:
repeating step (b) at selected intervals wherein the repeating is effective to track changes in the intensity of fluorescent emission in the subject over time to detect changes either in location or in number of cells that undergo cell death.

29-33. (canceled)
